(19) United States
(12) Patent Application Publication
(10) Pub. No.: US 2014/0329759 A1
(43) Pub. Date: Nov. 6, 2014

(54) POLYETHYLENE GLYCOL-MODIFIED INTEGRIN BLOCKER HM-3 AND USE THEREOF

(71) Applicant: Hanmei XU, (US)

(72) Inventors: Hanmei Xu, Nanjing (CN); Haimin Chang, Nanjing (CN); Zhian Kang, Nanjing (CN)

(21) Appl. No.: 14/359,462

(22) PCT Filed: Nov. 17, 2012
(86) PCT No.: PCT/CN2012/084788

§ 371 Date: May 20, 2014

(30) Foreign Application Priority Data
Nov. 21, 2011 (CN) 201110370529.9

(51) Int. Cl. A61K 47/48; A61K 38/10

(52) U.S. Cl. CPC A61K 47/48215; A61K 38/10
USPC 514/19.3; 530/326; 514/19.2

(57) ABSTRACT

The present invention involves the pharmaceutical field, including integrin antagonists, which have the capacities of inhibiting angiogenesis of tumors, binding integrin. These antagonists are a kind of polypeptide, which was modified by polyethylene glycol and after modification, it can be used to treat tumors. The sequence and structure of these antagonists is mPEG-SC₂₀ₖ-Ile-Val-Arg-Arg-Ala-Asp-Arg-Ala-Ala-Val-Pro-Gly-Gly-Gly-Gly-Arg-Gly-Asp, which demonstrates it is scientific, reasonable and feasible in tumor treatment and greatly expands the treatment spectrum. It can provide new ideas and perspectives for drug development and has significant social and market value.

### CROSS-REFERENCE TO RELATED APPLICATIONS

This application claims priority to PCT Application No. PCT/CN2012/084788, having a filing date of Nov. 17, 2012, based off of CN Application No. 201110370529.9, having a filing date of Nov. 21, 2011, the entire contents of which are hereby incorporated by reference.

### SEQUENCE LISTING

In accordance with the requirements of 37 C.F.R. §1.821(c), this application includes a Sequence Listing submitted on a compact disk in compliance with the requirements set forth in 37 C.F.R. §1.52(e). In accordance with the requirements set forth in 37 C.F.R. §1.52(e)(5), the contents of the Sequence Listing are hereby incorporated by reference.

### FIELD OF TECHNOLOGY

The present invention involves the pharmaceutical field, including an integrin inhibitor, which has the capacities of inhibiting angiogenesis of tumors, binding integrin. This inhibitor is a kind of polypeptide, which was modified by polyethylene glycol and after modification, it can be used to treat tumors.

### BACKGROUND

According to research, the growth of solid tumors relies on angiogenesis, which can not only provide nutrients and oxygen tumors need and excrete metabolites, but also the approach of distant metastasis. Therefore, blocking angiogenesis is a method that can prevent tumor growth and metastasis. And this theory stimulates extensive research on the pro-angiogenic molecules and anti-angiogenic molecules. Compared with the traditional medicine of tumors, the advantages of angiogenesis inhibitor are as follows: (1) Selectively affect vascular endothelial cells, and have a relatively lower systemic toxic side effect. (2) The target cells are vascular endothelial cells. Medicine is easy to reach and take effect. (3) Due to no or rare vascular endothelial cell mutant occurring, it is not easy to produce drug resistance, and the patients can have a long-term medication. (4) Can be combined with chemotherapy methods and reduce the toxicity of the latter.

Currently, the integrin antagonists exploited internationally has entered phase II clinical experiment, whereas there have been no similar products entering Chinese market. So it is necessary to develop such drugs of our own intellectual property. Now several antagonists are introduced. One of them is integrin antagonist polypeptide, and its sequence is Ile-Val-Arg-Arg-Ala-Asp-Arg-Ala-Ala-Val-Pro-Gly-Gly-Gly-Gly-Arg-Gly-Asp (SEQ ID NO 1), which contains integrin ligand sequences (Gly-Gly-Gly-Gly-Arg-Gly-Asp) (SEQ ID NO 2) and angiogenesis inhibition sequence (Ile-Val-Arg-Arg-Ala-Asp-Arg-Ala-Ala-Val-Pro) (SEQ ID NO 3). Integrin ligand sequences contain RGD sequences (Arg-Gly-Asp). Integrin antagonist polypeptide sequence can effectively bind integrin subtypes of tumor-specific expression and inhibit angiogenesis of tumors because of its angiogenesis inhibition sequence, eventually come to the results of preventing growth and metastasis of tumors. Previous research demonstrated that its targets are integrin ανβ3 and α5β1, and ανβ3 is the prime target. The polypeptide was testified to have better anti-tumor effect via repeated activity evaluation in vivo and vitro. It can significantly inhibit endothelial cell migration and tumor angiogenesis, thereby inhibit tumor growth. However, the half-life of the polypeptide is short. Clinically, this medicine is administered through intravenous infusion every day, so it brings the patients some pain.

According to report, it is a common method to solve the problems of continuous administration due to short half-time via modifying or alter molecular structure. Chemical modification is the most widely used. The chemical modifiers commonly used include polyethylene glycol (PEG), dextran, polyamino acids, polyanhydrides and so on. PEG has been approved as pharmaceutical auxiliary material and modifier by the U.S. Food and Drug Administration (FDA) because of the feature of non-toxicity, non-immunogenicity, and good water solubility. The molecular weight of protein drugs increases, while glomerular filtration rate decrease after PEG modification. PEG barrier protects protein from being hydrolyzed by protein proteolysis, meantime reduces the generation of neutralizing antibodies, which can extend the biological half-life of are protein drugs. Nowadays, there are a variety of protein drugs modified by PEG on sale. But PEG modification can also affect the biological activity of protein, and the degree of influence relates to modifier, modifying conditions and the nature of the protein itself For specific proteins, its best modification is determined by proteins modified by PEG and its biological activity. Although the study of PEG started recently, it has attracted a lot of attention from researchers.

### SUMMARY

The invention made a further research for mPEG-SC-Ile-Val-Arg-Arg-Ala-Asp-Arg-Ala-Ala-Val-Pro-Gly-Gly-Gly-Gly-Arg-Gly-Asp (SEQ ID NO 4), and discovered that it had therapeutic effect under the condition of decreasing the frequency of administration.

### Technical Programs

HM-3 is a kind of integrin inhibitor modified by polyethylene glycol and its sequence is mPEG-SC-Ile-Val-Arg-Arg-Ala-Asp-Arg-Ala-Ala-Val-Pro-Gly-Gly-Gly-Gly-Arg-Gly-Asp (SEQ ID NO 4). And the feature of this inhibitor is the range of molecular weight of mPEG-SC is 500-20000.The feature of HM-3 mentioned above is its molecular weight of mPEG-SC is 20000 and its treatment of tumor originated from stomach, skin, head and neck, thyroid, pancreas, lung, esophagus, breast, kidney, gall bladder, colon/rectum, ovary, uterus, cervix, prostate, bladder, testicular primary/secondary cancer or sarcoma.

### Beneficial Effects

1. In order to extend the half-life of Ile-Val-Arg-Arg-Ala-Asp-Arg-Ala-Ala-Val-Pro-Gly-Gly-Gly-Gly-Arg-Gly-Asp (SEQ ID NO 1), we used PEG of various molecular weights to modify this polypeptide and discovered that mPEG-SC_{2Qk}-HM-3 had the role of extending half-time of HM-3, meantime, the feature of no impact of activity in vivo and vitro. A product modified by polypeptide is a novel molecule. It usually produces different effects compared with the molecule without modification. This invention made many studies about therapeutic effect of mPEG-SC₂₀ₖ-HM-3 on many tumors and discovered that mPEG-SC₂₀ₖ-HM-3 played a positive role in inhibiting growth of many tumors, and expanded its social and economic value.

2. According to study, sequence of Ile-Val-Arg-Arg-Ala-Asp-Arg-Ala-Ala-Val-Pro (SEQ ID NO 3) had the function of inhibiting angiogenesis of tumors. The sequence of RGD was an important integrin ligand, so RGD-containing Gly-Gly-Gly-Gly-Arg-Gly-Asp (SEQ ID NO 2) could specifically recognize integrin. The integrin antagonist polypeptide could bind Gly-Gly-Gly-Gly-Arg-Gly-Asp (SEQ ID NO 2) sequence of RGD at C terminal of Ile-Val-Arg-Arg-Ala-Asp-Arg-Ala-Ala-Val-Pro (SEQ ID NO 3) which had the function of inhibiting angiogenesis of tumors. Meantime, polyethylene glycol modification was made at the N terminal of the integrin antagonist polypeptide, and eventually formed the sequence: mPEG-SC₂₀ₖ-Ile-Val-Arg-Arg-Ala-Asp-Arg-Ala-Ala-Val-Pro-Gly-Gly-Gly-Gly-Arg-Gly-Asp (SEQ ID NO 4), which contained PEG and 18 amino acid polypeptide. RGD sequence had the ability of integrin affinity and binding. Study demonstrated that the targets are integrin ανβ3 and α5β1, but the main target is ανβ3. RGD sequence contained angiogenesis inhibition sequence, eventually come to the results of preventing growth and metastasis of tumors. PEG is a kind of macromolecule polymer with unique physical and chemical properties. It has good biocompatibility and is non-toxic, non-antigenic. Main biological functions of protein or polypeptide drugs not only remain unchanged after undergoing PEG modification, but also can give protein or polypeptide drugs many excellent performances: (1) Increase stability, extend plasma half-life; (2) Reduce immunogenicity and antigenicity; (3) Reduce toxicity; (4) Reduce the possibility of degradation by hydrolytic enzymes and the rate of renal clearance; (5) Improve the distribution and dynamics of drug in the body. The target and anti-tumor activity of mPEG-SC₂₀ₖ polypeptide remain unchanged after modification, and extend half-time, reduce clearance and immunogenicity and antigenicity. Therefore, reduce the frequency of administration: from being administered once daily to once every 2-3 days. The results confirm that the integrin has advantages of significant anti-tumor effects, lower side effects, less dose and lower cost through many experiments, which demonstrated that the PEG modified integrin antagonist is scientific, reasonable, feasible and effective and can become treatment medicine of human anti-tumor. It can provide novel ideas and perspectives for future drug development, and expanded its social and economic value.

Half time of HM-3 before modification is 0.46 h, and the half time after modification by mPEG-SC20k is 20.13 h as indicated in the table below.

TABLE 1
The comparison of half-time of mPEG-
SC₂₀ₖ-HM-3 with HM-3 (t_{1/2}β)

| | | | AUC_{0-∞} | MRT_{0-∞} |
|---|---|---|---|---|
| Medicine | t_{1/2}β (h) | CL (L/h/kg) | (mg/L/h) | (h) |
| mPEG-SC₂₀ₖ- | 20.13 ± | 0.0071 ± | 4391.22 ± | 15.35 ± |
| HM-3 | 0.64 | 0.0012 | 3562.89 | 1.07 |
| HM-3 | 0.46 ± | 1.38 ± | 25.63 ± | 0.036 ± |
| | 0.12 | 0.15 | 9.76 | 0.002 |

### BRIEF DESCRIPTION

Some of the embodiments will be described in detail, with reference to the following figures, wherein like designations denote like members, wherein:

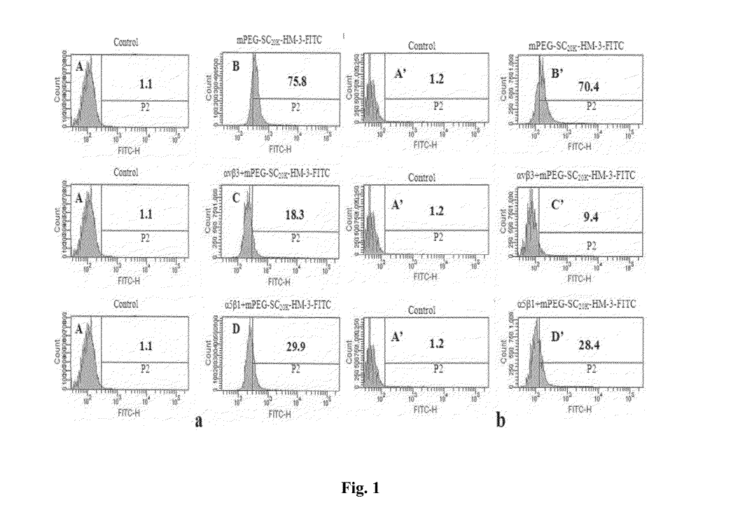

FIG. 1 The integrin antagonist polypeptide binded target via flow cytometry experiments. (a) showed the first test, and (b) was repeated test;

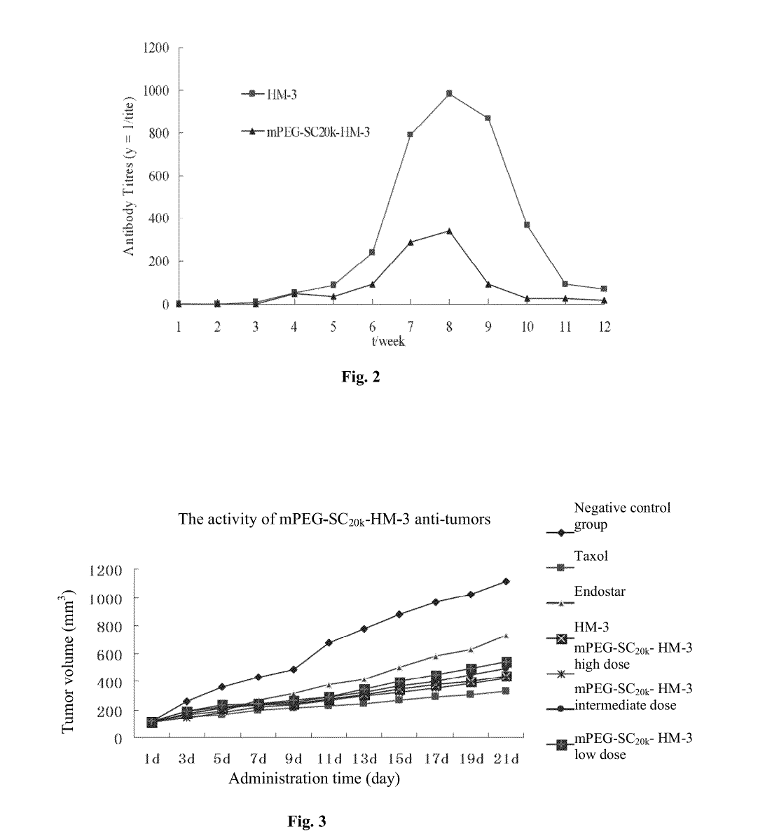

FIG. 2 The comparison of the integrin antagonist polypeptide with HM-3 immunogenicity

FIG. 3 The effect of tumor growth inhibition induced by integrin antagonist polypeptide on human esophageal cancer Ecl09 xenograft tumors in nude mice;

FIG. 4 The effect of tumor growth inhibition induced by integrin antagonist polypeptide on human nasopharyngeal cancer CNE xenograft tumors in nude mice;

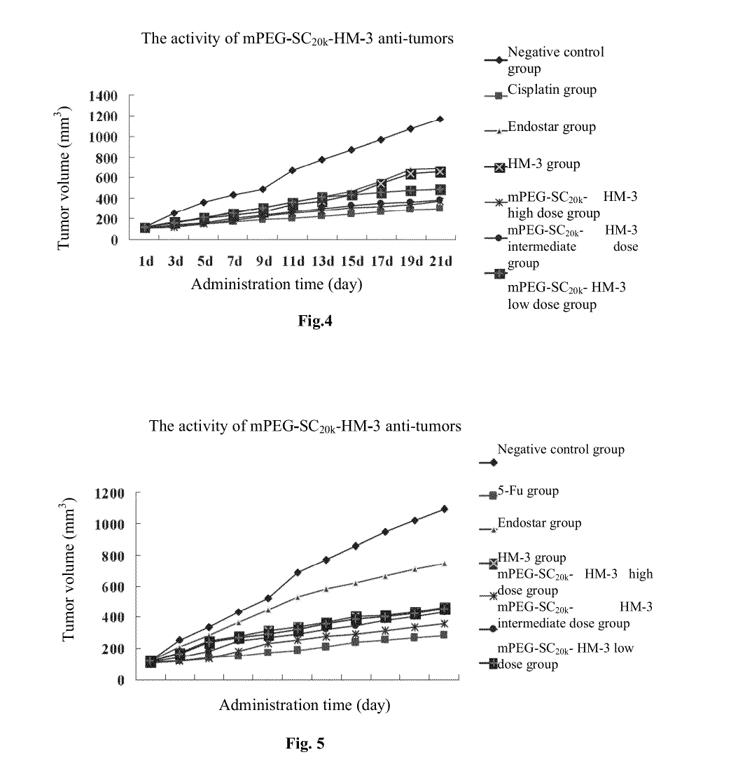

FIG. 5 The effect of tumor growth inhibition induced by integrin antagonist polypeptide on human Thyroid cancer SW-579 xenograft tumors in nude mice;

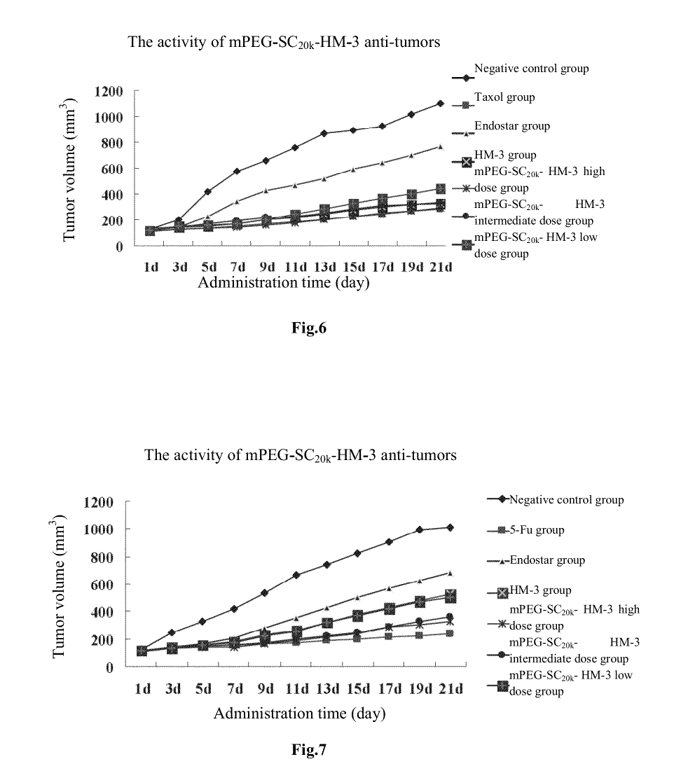

FIG. 6 The effect of tumor growth inhibition induced by integrin antagonist polypeptide on human gastric cancer MGC803 xenograft tumors in nude mice;

FIG. 7 The effect of tumor growth inhibition induced by integrin antagonist polypeptide on human pancreatic cancer SW-1990 xenograft tumors in nude mice;

FIG. 8 The effect of tumor growth inhibition induced by integrin antagonist polypeptide on human lung cancer H460 xenograft tumors in nude mice;

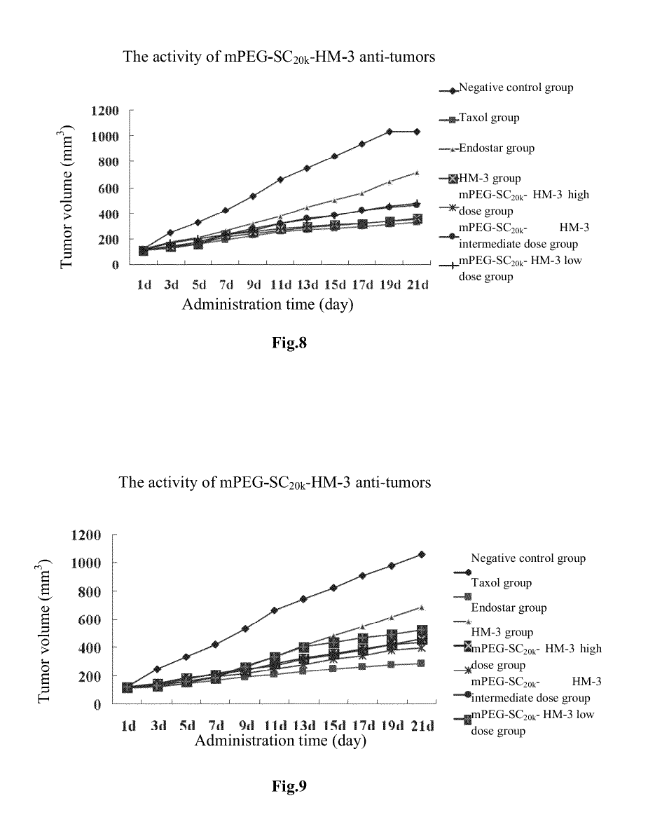

FIG. 9 The effect of tumor growth inhibition induced by integrin antagonist polypeptide on human breast cancer MDA-MB-231 xenograft tumors in nude mice;

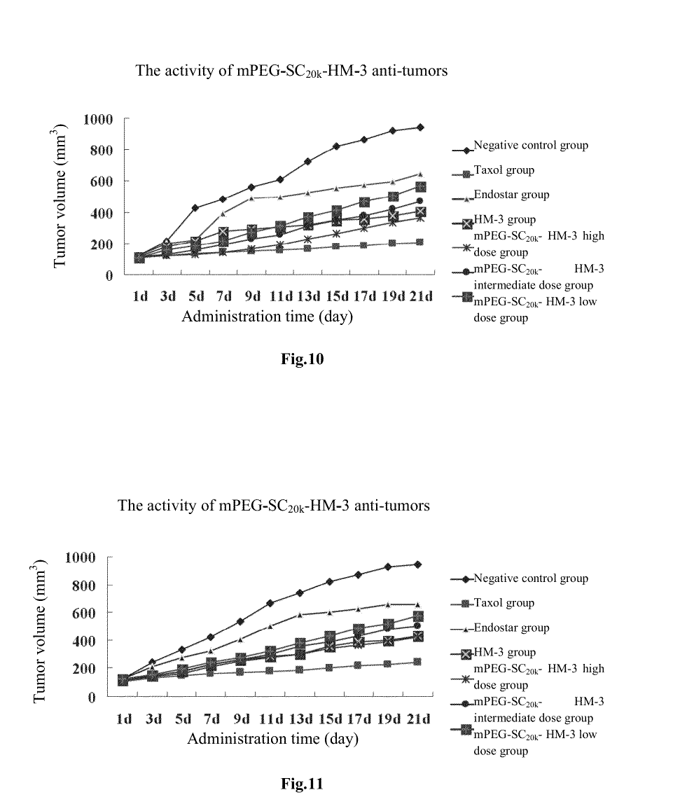

FIG. 10 The effect of tumor growth inhibition induced by integrin antagonist polypeptide on human gallbladder cancer GBC-SD xenograft tumors in nude mice;

FIG. 11 The effect of tumor growth inhibition induced by integrin antagonist polypeptide on human kidney cancer A498 xenograft tumors in nude mice;

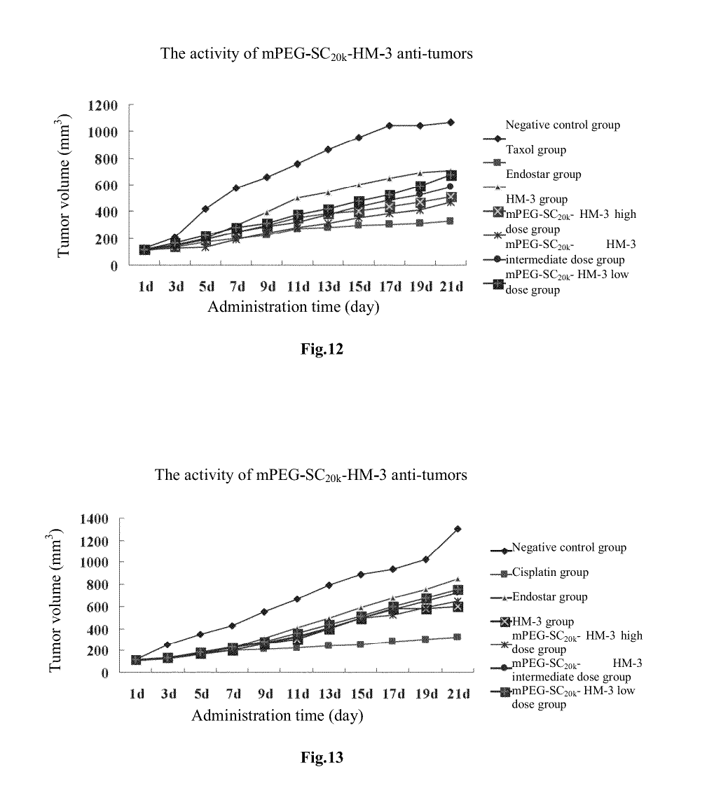

FIG. 12 The effect of tumor growth inhibition induced by integrin antagonist polypeptide on human colon cancer HT-29 xenograft tumors in nude mice;

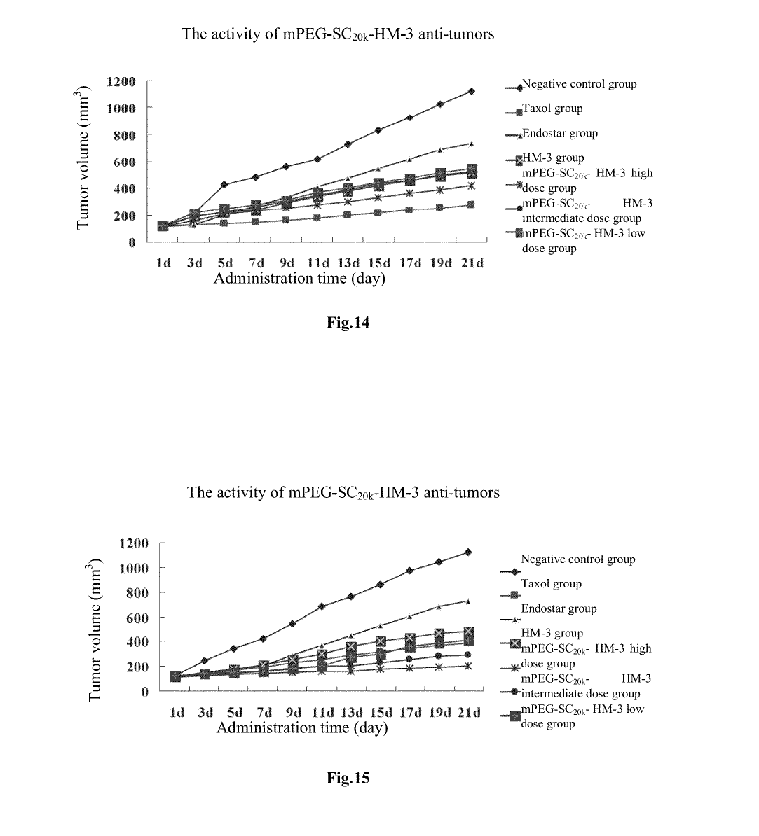

FIG. 13 The effect of tumor growth inhibition induced by integrin antagonist polypeptide on human ovarian cancer SK-OV-3 xenograft tumors in nude mice;

FIG. 14 The effect of tumor growth inhibition induced by integrin antagonist polypeptide on human endometrial cancer HHUA xenograft tumors in nude mice;

FIG. 15 The effect of tumor growth inhibition induced by integrin antagonist polypeptide on human cervical cancer HeLa xenograft tumors in nude mice;

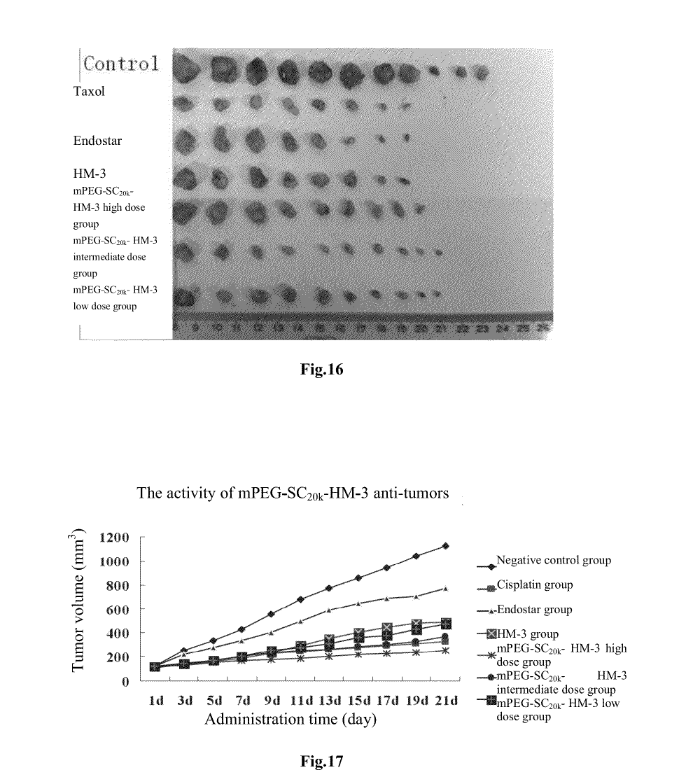

FIG. 16 The tumor picture of human cervical cancer HeLa xenograft tumors in nude mice inhibited by integrin antagonist polypeptide;

FIG. 17 The effect of tumor growth inhibition induced by integrin antagonist polypeptide on human prostate cancer DU-145 xenograft tumors in nude mice;

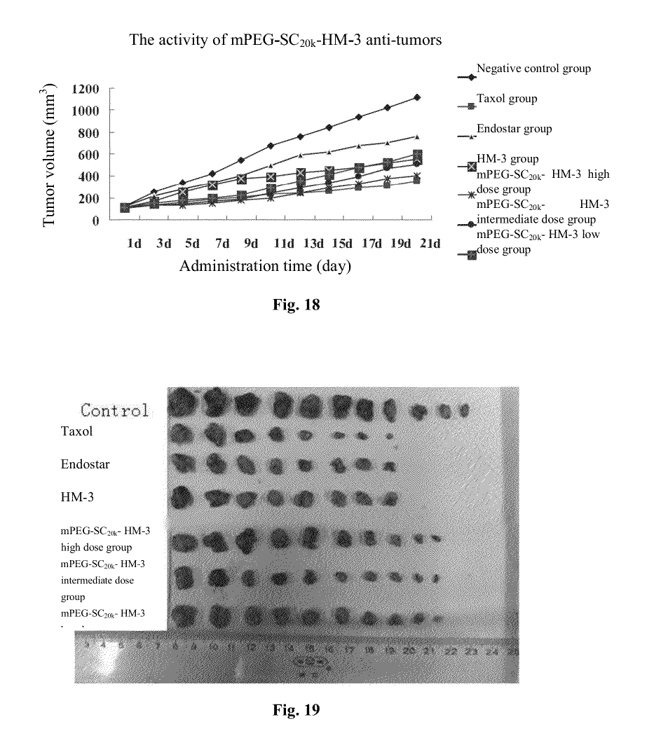

FIG. 18 The effect of tumor growth inhibition induced by integrin antagonist polypeptide on human bladder cancer HT1376 xenograft tumors in nude mice;

FIG. 19 The tumor picture of human bladder cancer HT1376 xenograft tumors in nude mice inhibited by integrin antagonist polypeptide;

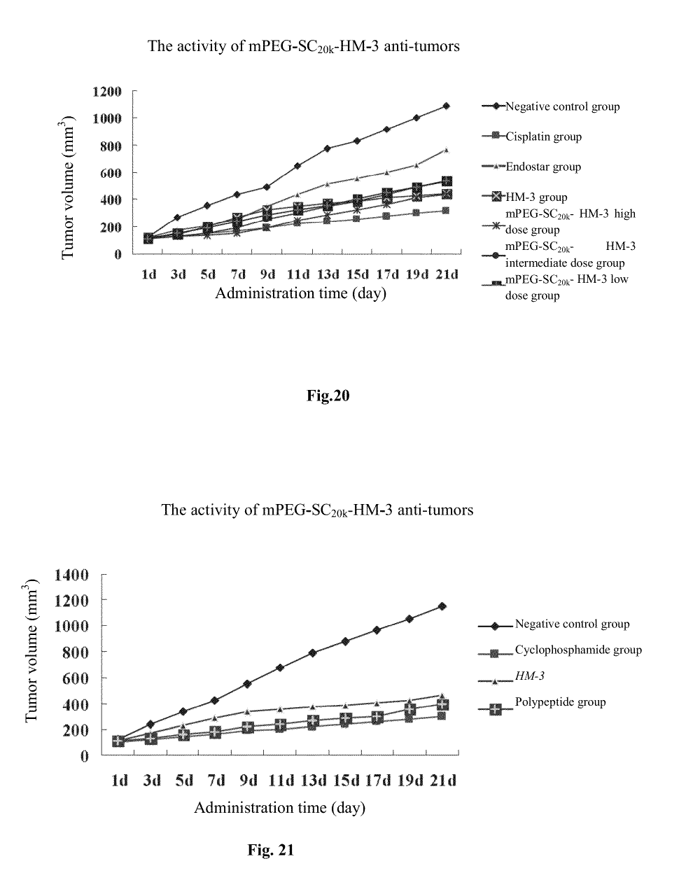

FIG. 20 The effect of tumor growth inhibition induced by integrin antagonist polypeptide on human testicular cancer 5637 xenograft tumors in nude mice; and

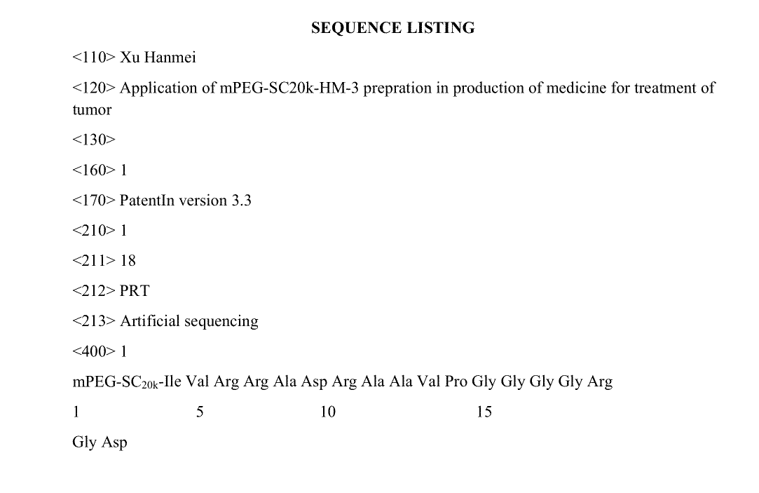

FIG. 21 The effect of tumor growth inhibition induced by integrin antagonist polypeptide on sarcoma HT-1080 xenograft tumors in nude mice.

### SPECIFIC OPERATING METHODS

### EXAMPLE 1

### Integrin Antagonist HM-3

The method of synthesizing solid-state of Ile-Val-Arg-Arg-Ala-Asp-Arg-Ala-Ala-Val-Pro-Gly-Gly-Gly-Gly-Arg-Gly-Asp (SEQ ID NO 1):Fmoc-Ile-wang resin or Fmoc-Ile-CTC resin is regarded as a raw material and then protect amino acids. Dipeptides to eighteen peptide are sequentially connected, then washed thoroughly, followed by peptide cut and post-management to obtain a crude product of HM-3. The crude product was purified. At first, it is dissolved, and then purified twice by preparative HPLC, and finally lyophilized to give a pure product. Specific steps are as follows:

### 1. Synthesis

Weigh Fmoc-Ile-Wang resin 14.7 g, and then pour it into a glass sand core reaction column of 1 L volume. The CH2Cl2 147 ml was added to make resin full expansion.

Uncapping: After the solution of uncapping 25 ml with hexahydropyridine/DMF is sealed, keep it in the shaker for 5 minutes under the temperature of room temperature. Then the uncapping solution is drained, and washed once again with DMF in the middle and then add 20% uncapping solution 25 ml with a reaction of 15 minutes;

Washing: Drained the uncapping solution, and then is washed six times with the DMF resin, drained, and then take 20 resin into a small test tube, add test reagent, and heated under the temperature of 115° C. for 3 minutes.

Condensating: Weigh protective amino acids and HOBt 2.025 g, then be dissolved in 15 ml DMF and 2.33 ml DIC, then pour it into the reactor lasting about 1.5 hours, the temperature is controlled at about 34° C.

Washing: Drain the reaction solution, and wash the resin with DMF three times, drain again, and then put 10-20 resin particles into a small test tube, add test toner and heat it at 115° C. 3-5 min.

Cutting: Put the drained resin into round bottom flask of 500 mL volume, and then add 90% cleavage solution of 300 mL (phenol:thioanisole:EDT=90:3:3:2:2), and seal for 2 hours. Separate polypeptide from resin with a frit funnel.

Postconditioning: At first add anhydrous ether to cleavage solution to precipitate the polypeptide, then centrifuge, then the supernatant was discarded, and then polypeptide is washed with anhydrous ether six times. Finally, drain it to gain crude peptide 9.5 g

### 2. Purification

Dissolving: Weigh ID-18 crude product exactly, then add purified water to the appropriate configuration of 10 g/l solution. Ultrasonic stirs until a clear solution without particulate comes up.
Filtrating: The ID-18 solution is filtrated with 0.45 um membrane of sand core filter.
Producing:

### The Primary Purification

Balancing: Configure the solution of 5% acetonitrile and 95% aqueous trifluoroacetic acid. Rinse 10 min at a flow rate 80 ml/min.

Loading: Load with the infusion pump, flow rate 80 ml/min. And collect baseline and solution which is greater in the UV wavelength absorption of 220 nm than 200 mv to detect whether there are samples out.

Eluting: Gradient of elution

| Time | Velocity of flow | Acetonitrile | Aqueous trifluoroacetic |
|---|---|---|---|
| (min) | (ml/min) | % | acid % |
| 0 | 80 | 8 | 92 |
| 40 | 80 | 18 | 82 |

Collect solution which is stronger in the UV wavelength absorption of 220 nm than 200 mvUV. The purity of greater than 95% is regarded as a peak and ready to do a secondary separation and purification.

### The Secondary Purification

Balancing: Configure the solution of 5% acetonitrile+95% aqueous acetic acid. Rinse 10 min at a flow rate 80 ml/min.

Loading: Load the primary peak with infusion pump after evaporating the organic solvent. And collect baseline and solution which is greater in the UV wavelength absorption of 220 nm than 200 mv to detect whether there are samples out.

Eluting: Gradient of elution

| Time | Velocity of flow | Acetonitrile | aqueous acetic |
|---|---|---|---|
| (min) | (ml/min) | % | acid % |
| 0 | 80 | 5 | 95 |
| 40 | 80 | 15 | 85 |

Collect the solution which is stronger in the UV wavelength absorption of 220 nm than 200 mv. The purity of greater than 99% is regarded as qualification. Concentrating, filtering and freeze-drying: Concentrate the qualified solution with a rotary evaporator under 37° C. and remove residual solvent and water. Finally filter it with 0.22 um filter membrane and put the filtrate into the freeze-dried pan, then freeze and dry it with a freeze dryer to give pure product.

### EXAMPLE 2

### Steps of Polyethylene Glycol Modifying Polypeptide

The reaction of mPEG-SC20K and the HM-3

Weigh 2 g mPEG-SC20k and 106.24 mg HM-3 (molar ratio 1.5:1) respectively. Both of them are placed in 40 ml-100 ml pH 5-8.5 PBS buffer solution at the conditions at 4° C. overnight and allow them to react. PEG-SC500-20000 can be connected as described in Example 2 to produce modified polypeptides

### EXAMPLE 3

### The Steps of Separation and Purification

### 1. Separation

The sample after the reaction is purified through semi-preparative HPLC (HPLC, BIO-RAD) and purification conditions are as follows:
Mobile phase: ACN (+0.1% TFA), H₂O (+0.1% TFA); ACN linear gradient: 40% -95%;
Flow rate: 2 ml/min; Running time: 12 min;
Loading volume: 1.0 ml; detecting wave length: 220 nm
Semi-preparative column: YMC, 250 mm×10 mm (5 μm packing).
   In the process of peaks of the peak, the product was collected by centrifugation tube

### 2. Purification

The collected products through HPLC are frozen in the cryogenic freezer at −70° C. overnight, then freeze and dry them through the freeze dryer until the products become white powder (30 h or so). Gain lyophilized product, weigh and record the weight of the products, and then save them in the −20° C. refrigerator and make identification.

### 1. Analysis of Purity of the Products

The products are lyophilized and analyzed by analytical HPLC. The conditions of purity analysis are as follows:
Mobile phase: ACN (+0.1% TFA), H₂O (+0.1% TFA); ACN linear gradient: 10%-100%;
Flow rate: 1 ml/min;
Running time: 15 min;
Sample volume: 20 μl; detection wavelength: 220 nm
Analytical Column: Beijing innovation Tongheng, 250 mm×4.6 mm (5 μm filler)

### 2. SDS-PAGE Analysis of Modified Products

Basic operation reference “Molecular Cloning (The second edition).” Concentration of stacking gel is 5%, and separating gel 10%, concentrated voltage 80 volts, separation voltage 120 volts. The bands of samples are stained with Bal2 first after electrophoresis especially for the portion containing PEG; Coomassie brilliant blue R250 as a Marker stains part of the protein. Place it into bleaching solution after staining to the effect of background transparent and then analyze the results through scanning The following takes mPEG20000 modified polypeptide integrin antagonist polypeptide (mPEG-SC20k-HM-3.for example to make description. Integrin antagonist polypeptide described in example is the integrin antagonist polypeptide modified by mPEG20000.

### EXAMPLE 4

### The Study of Pharmacokinetics of mPEG-SC20k-HM-3 in Rats

SD rats were randomly divided into six groups with the same number for male and female. Take three groups were administered intravenously integrin antagonist polypeptide with a high dose of 52 mg/kg (equivalent to HM-3 4.2 mg/kg), an intermediate dose of 26 mg/kg (equivalent to HM-3 2.1 mg/kg), a low-dose of 13 mg/kg (equivalent to HM-3 1.05 mg/kg). The other 3 groups were injected HM-3 with a high dose of 4.2 mg/kg, an intermediate dose of 2.1 mg/kg, a low doses of 1.05 mg/kg. Collected whole blood 0.5 ml once from the orbital venous plexus after 0.5 h, 1 h, 2 h, 3 h, 6 h, 12 h, 24 h, 48 h, 72 h, 96 h, 108 h, 132 h of drug administration, and applied heparin to get the effect of anti-coagulation. 12000 rpm/2 min centrifuged plasma. Draw supernatant 200 μl and 80° C. preheated PBS (0.05M pH 7.4) buffer 600 μl and mixed. Bath in the 80° C. water for 30 min. Centrifuged 2 min at 12000 rpm, and collected the supernatant, and stored in the condition of −20° C. Dissolved at room temperature, measured integrin antagonist peptide plasma concentration by ELISA.

### The Comparison of Pharmacokinetic Parameters of mPEG-SC20k-HM-3 and HM-3 in SD Rats (t_{1/2}β Halt-Time, CL the Rate of Plasma Clearance, AUD Area Under the Curve, MRT the Average Residence Time)

| | | | AUC_{0-∞} | MRT_{0-∞} |
|---|---|---|---|---|
| Drugs | t_{1/2}p (min) | CL (L/h/kg) | (mg/L/h) | (min) |
| mPEG-SC₂₀ₖ- | 1207.80 ± | 0.0071 ± | 4391.22 ± | 921.18 ± |
| HM-3 | 38.40 | 0.0012 | 3562.89 | 64.29 |
| HM-3 | 27.66 ± | 1.38 ± | 25.63 ± | 2.17 ± |
| | 7.37 | 0.15 | 9.76 | 0.13 |

As known from Table 1, compared with the HM-3, mPEG-SC₂₀ₖ-HM-3 has a significantly longer half-life and lower plasma clearance. The above experimental data have demonstrated PEG-modified protein can significantly improve the feature of pharmacokinetics of polypeptide drug in rats

### EXAMPLE 5

### Analyzing the Combination of mPEG-SC₂₀ₖ-HM-3 and Targets Through Flow Cytometry

(1) Bel-7402 tumor cells and MCF-7 fusion cultured to 80% after the 24-well plate, trypsinized collected, washed with ice-cold PBS twice, the cells were labeled prior containing 1% BSA resuspended in PBS 30 min.
(2) 2 ml mouse anti-human ανβ3 function-blocking monoclonal antibody (1.0 μg/μl, 1:200), mouse anti-human 2 μl α5β1 function-blocking monoclonal antibody (1.0 μg/μl, 1:200) and the cell suspension were incubated at 4° C. 1.5 h.
(3) collected the labeled cells and washed with ice-cold PBS twice, followed by 100 μl FITC labeled polypeptide modified mPEG-SC20k-HM-3 (2 mg/ml) and the cell suspension were incubated at 4° C. incubate 1.5 h
(4) After labeled, the cells were collected and washed with ice-cold PBS 2 times, followed by resuspension with 400 μl PBS and analyzed by flow cytometry, and detected intensity of the FITC fluorescence through channel FL1.

FIG. 1 showed mPEG-SC20k-HM-3 was able to bind ανβ3 and α5β1of integrin, but the main target was still ανβ3. It demonstrated that the main targets of HM-3 were not changed after PEG modifying, and the active sites are not covered by PEG.

### EXAMPLE 6

### The Assay of Immunogenicity of mPEG-SC₂₀ₖ-HM-3 and HM-3

BALB/c white mice were randomly divided into 2 groups, and the same number of male and female. 36 mg/kg mPEG-SC₂₀ₖ-HM-3 and 3.0 mg/kg HM-3 were administrated intravenously through tail vein, respectively, lasting 8 weeks, and collected blood from the orbital venous plexus once a week during 1-12 weeks. Centrifuged 2 min at 12000 rpm, collected the supernatant, and stored in the condition of −20° C. Dissolved at room temperature, 0.1 ml was used to measure antibody titers in serum by ELISA.

### Group Settings:

Group 1 The effective dose of HM-3 was 3.0 mg/kg. Administration: once a day, 6 rats (male, 3; female, 3)

Group 2 The effective dose of mPEG-SC₂₀ₖ-HM-3 was 36 mg/kg. Administration: once two days, 6 rats (male,3; female, 3)

FIG. 2 showed that antibody appeared at the third week of administration in HM-3 group, whereas lower titer antibody was detected at the fifth week of administration in mPEG-SC₂₀ₖ-HM-3 group. The antibody titer of integrin antagonist polypeptide group was significantly lower than HM-3 group at any time points. After cession of administration, antibody titer decreased gradually, and the antibody in integrin antagonist polypeptide group could not detect at the twelfth week of administration, which demonstrated that PEG modification could significantly reduce the immunogenicity of HM-3 in vivo.

### EXAMPLE 7

### The Experiment of Tumor Growth Inhibition Induced by mPEG-SC₂₀ₖ-HM-3 on Human Esophageal Cancer Ecl09 Xenograft Tumors in Nude Mice

Picked the tumor tissue of vigorous growth and was cut into pieces of about 1.5 mm³. Under sterile conditions, the tumor tissue was inoculated subcutaneously on the right of nude mice. Tumor diameter was measured with a vernier caliper. The animals which had tumors with a size of 100-200 mm³ were randomly divided into groups. The effect of polypeptide on anti-tumor was dynamically observed through the method of measuring tumor diameter. The times of measuring tumor diameter were once 2 days, and meantime, weighed mice. The mice in experimental group were infused polypeptide through tail vein, once 3 days. The mice in the placebo group were injected saline with the same volume. Tumor volume formula:

TV=0.52×a×b²

a, b respectively represent the length and width. According to the result of measurement, calculate the relative tumor volume. Evaluation of antitumor activity is relative tumor proliferation rate T/C (%). The formula is as follows:

T/C(%)⁻T_{RTV}/C_{RTV}×100%

T_{RTV}: Treatment group RTV; C_{RTV}: negative control group RTV

TABLE 2
The experiment of tumor growth inhibition induced by mPEG-SC₂₀ₖ-HM-
3 on human esophageal cancer Ec109 xenograft tumors in nude mice

| | | | Primary | | Final | | Tumor | Rate of |
|---|---|---|---|---|---|---|---|---|
| | Dose | Frequency of | weight | Primary | weight | Final | weight | inhibiting |
| Groups | (mg/kg) | administration | (g) | numbers | (g) | numbers | (g) | tumors (%) |
| Negative | — | Once 1 day | 21.43 | 12 | 21.22 | 12 | 0.926 | — |
| control | | | | | | | | |
| Taxol | 10 | Once 2 days | 20.56 | 8 | 20.24 | 8 | 0.276 | 70.19% |
| Endostar | 2.5 | Once 1 day | 21.55 | 8 | 21.26 | 8 | 0.602 | 35.0% |
| HM-3 | 1.5 | Once 3 days | 21.42 | 8 | 20.74 | 8 | 0.365 | 60.57% |
| mPEG-SC_{20K}-HM-3 | 36.7 | Once 3 days | 22.20 | 8 | 21.75 | 8 | 0.183 | 62.24%* |
| high | | | | | | | | |
| mPEG-SC_{20K}-HM-3 | 18.75 | Once 3 days | 20.45 | 8 | 19.93 | 8 | 0.318 | 55.66% |
| intermediate | | | | | | | | |
| mPEG-SC_{20K}-HM-3 | 9.38 | Once 3 days | 21.22 | 8 | 20.38 | 8 | 0.406 | 50.16% |
| low | | | | | | | | |

Results: shown in table 2 and FIG. 3. Taxol group: 10 mg/kg, the rate of inhibiting tumor of nude mice with human esophageal cancer Ecl09 is 70.19%; Endostar group: 2.5 mg/kg, the rate of inhibiting tumor of nude mice with human esophageal Ecl09 is 35.0%; HM-3 group: 1.5 mg/kg, the rate of inhibiting tumor of nude mice with human esophageal Ecl09 is 60.57%; The rates of inhibiting tumor of nude mice with human esophageal Ecl09 in groups of high, intermediate and low dose of polypeptide are 62.24%, 55.66%, 50.16% respectively.

The results of experiment of tumor growth inhibition induced by mPEG-SC₂₀ₖ-HM-3 on human esophageal cancer Ecl09 xenograft tumors in nude mice demonstrated that compared with negative control group, mPEG-SC_{20K}-HM-3 36.7 mg/kg played a significant inhibition role in growth of human esophageal cancer Ecl09. *P<0.05 (there is significant difference compared with negative group).

### EXAMPLE 8

### The Experiment of Tumor Growth Inhibition Induced by mPEG-SC₂₀ₖ-HM-3 on Human Nasopharyngeal Cancer CNE Xenograft Tumors in Nude Mice

Picked the tumor tissue of vigorous growth and was cut into pieces of about 1.5 mm³. Under sterile conditions, the tumor tissue was inoculated subcutaneously on the right of nude mice. Tumor diameter was measured with a vernier caliper. The animals which had tumors with a size of 100-200 mm³ were randomly divided into groups. The effect of polypeptide on anti-tumor was dynamically observed through the method of measuring tumor diameter. The times of measuring tumor diameter were once 2 days, and meantime, weighed mice. The mice in experimental group were infused polypeptide through tail vein, once 3 days. The mice in the placebo group were injected saline with the same volume. Tumor volume formula:

TV=0.52×a×b²

a, b respectively represent the length and width. According to the result of measurement, calculate the relative tumor volume. Evaluation of antitumor activity is relative tumor proliferation rate T/C (%). The formula is as follows: T/C (%)⁼T_{RTV}/C_{RTV}×100%
T_{RTV}: Treatment group RTV; C_{RTV}: negative control group RTV

TABLE 3
The effect of tumor growth inhibition induced by mPEG-SC₂₀ₖ-HM-3
on human nasopharyngeal cancer CNE xenograft tumors in nude mice

| | | | Primary | | Final | | Tumor | Rate of |
|---|---|---|---|---|---|---|---|---|
| | Dose | Frequency of | weight | Primary | weight | Final | weight | inhibiting |
| Groups | (mg/kg) | administration | (g) | numbers | (g) | numbers | (g) | tumors (%) |
| Negative | — | Once 1 day | 22.78 | 12 | 22.37 | 12 | 1.201 | — |
| control | | | | | | | | |
| Cisplatin | 10 | Twice 1 week | 22.35 | 8 | 22.33 | 7 | 0.320 | 73.36% |
| Endostar | 2.5 | Once 1 day | 22.43 | 8 | 22.39 | 8 | 0.757 | 37.01% |
| HM-3 | 1.5 | Once 3 days | 22.60 | 8 | 22.85 | 8 | 0.468 | 61.03% |
| mPEG-SC₂₀ₖ-HM-3 | 36.7 | Once 3 days | 22.16 | 8 | 22.15 | 8 | 0.382 | 68.22%* |
| high | | | | | | | | |
| mPEG-SC₂₀ₖ-HM-3 | 18.75 | Once 3 days | 22.75 | 8 | 24.00 | 8 | 0.406 | 66.19% |
| intermediate | | | | | | | | |
| mPEG-SC₂₀ₖ-HM-3 | 9.38 | Once 3 days | 23.02 | 8 | 23.70 | 8 | 0.537 | 55.32% |
| low | | | | | | | | |

Results: shown in table 3 and FIG. 4. Cisplatin group: 10 mg/kg, the rate of inhibiting tumor of nude mice with human nasopharyngeal cancer CNE is 73.36%, but significantly affects the weight of experimental animals; Endostar group: 2.5 mg/kg, the rate of inhibiting tumor of nude mice with human nasopharyngeal cancer CNE is 37.01%; HM-3 group: 1.5 mg/kg, the rate of inhibiting tumor of nude mice with human nasopharyngeal cancer CNE is 61.03%; The rates of inhibiting tumor of nude mice with human nasopharyngeal cancer CNE in groups of high, intermediate and low dose of polypeptide are 68.22%, 66.19%, 55.32% respectively, but there is no significant influence on the weight of experimental mice.

The results of experiment of tumor growth inhibition induced by mPEG-SC₂₀ₖ-HM-3 on human nasopharyngeal cancer CNE xenograft tumors in nude mice demonstrated that compared with negative control group, mPEG-SC_{20K}-HM-3 36.7 mg/kg played a significant inhibition role in growth of human nasopharyngeal cancer CNE; compared with positive cisplatin group, there was no significant influence on the weight of experimental mice, and no obvious toxic and side effects. *P<0.05 (there is significant difference compared with negative group)

### EXAMPLE 9

### The Experiment of Tumor Growth Inhibition Induced by mPEG-SC₂₀ₖ-HM-3 on Human Thyroid Cancer SW-579 Xenograft Tumors in Nude Mice

Picked the tumor tissue of vigorous growth and was cut into pieces of about 1.5 mm³. Under sterile conditions, the tumor tissue was inoculated subcutaneously on the right of nude mice. Tumor diameter was measured with a vernier caliper. The animals which had tumors with a size of 100-200 mm³ were randomly divided into groups. The effect of polypeptide on anti-tumor was dynamically observed through the method of measuring tumor diameter. The times of measuring tumor diameter were once 2 days, and meantime, weighed mice. The mice in experimental group were infused polypeptide through tail vein, once 3 days. The mice in the placebo group were injected saline with the same volume. Tumor volume formula:

TV=0.52×a×b²

a, b respectively represent the length and width. According to the result of measurement, calculate the relative tumor volume. Evaluation of antitumor activity is relative tumor proliferation rate T/C (%). The formula is as follows: T/C (%)⁼T_{RTV}/C_{RTV}×100%
T_{RTV}: Treatment group RTV; C_{RTV}: negative control group RTV

TABLE 4
The effect of tumor growth inhibition induced by mPEG-SC₂₀ₖ-HM-
3 on human thyroid cancer SW-579 xenograft tumors in nude mice

| | | | Primary | | Final | | Tumor | Rate of |
|---|---|---|---|---|---|---|---|---|
| | Dose | Frequency of | weight | Primary | weight | Final | weight | inhibiting |
| Groups | (mg/kg) | administration | (g) | numbers | (g) | numbers | (g) | tumors (%) |
| Negative | — | Once 1 day | 21.34 | 12 | 21.23 | 12 | 1.253 | — |
| control | | | | | | | | |
| 5-Fu | 10 | Once 1 day | 21.56 | 8 | 21.32 | 7 | 0.320 | 74.50% |
| Endostar | 2.5 | Once 1 day | 21.67 | 8 | 20.39 | 8 | 0.850 | 32.20% |
| HM-3 | 1.5 | Once 1 day | 22.76 | 8 | 22.70 | 8 | 0.530 | 57.70% |
| mPEG-SC₂₀ₖ-HM-3 | 36.7 | Once 3 day | 22.32 | 8 | 21.76 | 8 | 0.406 | 67.63%* |
| high | | | | | | | | |
| mPEG-SC₂₀ₖ-HM-3 | 18.75 | Once 3 day | 22.90 | 8 | 22.42 | 8 | 0.494 | 60.56% |
| intermediate | | | | | | | | |
| mPEG-SC₂₀ₖ-HM-3 | 9.38 | Once 3 day | 21.58 | 8 | 21.28 | 8 | 0.521 | 58.42% |
| low | | | | | | | | |

Results: shown in table 4 and FIG. 5. 5-Fu group: 10 mg/kg, the rate of inhibiting tumor of nude mice with human thyroid cancer SW-579 is 74.50%, but 5-Fu has greater toxicity and leads to the weight loss and death of experimental animals; Endostar group: 2.5 mg/kg, the rate of inhibiting tumor of nude mice with human thyroid cancer SW-579 is 32.20%; HM-3 group: 1.5 mg/kg, the rate of inhibiting tumor of nude mice with human thyroid cancer SW-579 is 57.70%; The rates of inhibiting tumor of nude mice with human thyroid cancer SW-579 in groups of high, intermediate and low dose of polypeptide are 67.63%, 60.56%, 58.42%, respectively, but there is no significant influence on the weight of experimental mice.

The results of experiment of tumor growth inhibition induced by mPEG-SC₂₀ₖ-HM-3 on human thyroid cancer SW-579 xenograft tumors in nude mice demonstrated that compared with negative control group, mPEG-SC_{20K}-HM-3 36.7 mg/kg played a significant inhibition role in growth of human thyroid cancer SW-579; compared with positive 5-Fu group, there was no significant influence on the weight of experimental mice, and no obvious toxic and side effects. *P<0.05(there is significant difference compared with negative group).

### EXAMPLE 10

### The Experiment of Tumor Growth Inhibition Induced by mPEG-SC₂₀ₖ-HM-3 on Human Gastric Cancer MGC803 Xenograft Tumors in Nude Mice

Picked the tumor tissue of vigorous growth and was cut into pieces of about 1.5 mm³. Under sterile conditions, the tumor tissue was inoculated subcutaneously on the right of nude mice. Tumor diameter was measured with a vernier caliper. The animals which had tumors with a size of 100-200 mm³ were randomly divided into groups. The effect of polypeptide on anti-tumor was dynamically observed through the method of measuring tumor diameter. The times of measuring tumor diameter were once 2 days, and meantime, weighed mice. The mice in experimental group were infused polypeptide through tail vein, once 3 days. The mice in the placebo group were injected saline with the same volume. Tumor volume formula:

TV=0.52×a×b²

a, b respectively represent the length and width. According to the result of measurement, calculate the relative tumor volume. Evaluation of antitumor activity is relative tumor proliferation rate T/C (%). The formula is as follows: T/C (%)⁼T_{RTV}/C_{RTV}×100%
T_{RTV}: Treatment group RTV; C_{RTV}: negative control group RTV

TABLE 5
The effect of tumor growth inhibition induced by mPEG-SC₂₀ₖ-HM-
3 on human gastric cancer MGC803 xenograft tumors in nude mice

| | | | Primary | | Final | | Tumor | Rate of |
|---|---|---|---|---|---|---|---|---|
| | Dose | Frequency of | weight | Primary | weight | Final | weight | inhibiting |
| Groups | (mg/kg) | administration | (g) | numbers | (g) | numbers | (g) | tumors (%) |
| Negative control | — | Once 1 day | 22.32 | 12 | 22.15 | 12 | 0.723 | — |
| Taxol | 10 | Once 2 days | 22.14 | 8 | 22.05 | 8 | 0.187 | 74.12% |
| Endostar | 2.5 | Once 1 day | 23.22 | 8 | 23.13 | 8 | 0.504 | 30.29% |
| HM-3 | 1.5 | Once 3 days | 22.52 | 8 | 22.37 | 8 | 0.214 | 70.40% |
| mPEG-SC₂₀ₖ⁻HM-3high | 36.7 | Once 3 days | 22.67 | 8 | 22.24 | 8 | 0.192 | 73.42%* |
| mPEG-SC₂₀ₖ⁻HM-3 intermediate | 18.75 | Once 3 days | 22.76 | 8 | 22.32 | 8 | 0.218 | 69.86% |
| mPEG-SC₂₀ₖ⁻HM-3 low | 9.38 | Once 3 days | 22.81 | 8 | 22.44 | 8 | 0.292 | 59.57% |

Results: shown in table 4 and FIG. 5. Taxol group: 10 mg/kg, the rate of inhibiting tumor of nude mice with human gastric cancer MGC803 is 74.12%; Endostar group: 2.5 mg/kg, the rate of inhibiting tumor of nude mice with human gastric cancer MGC803 is 32.29%; HM-3 group: 1.5 mg/kg, the rate of inhibiting tumor of nude mice with human gastric cancer MGC803 is 70.40%; The rates of inhibiting tumor of nude mice with human gastric cancer MGC803 in groups of high, intermediate and low dose of polypeptide are 73.42%, 69.86%, 59.57%, respectively.

The results of experiment of tumor growth inhibition induced by mPEG-SC₂₀ₖ-HM-3 on human gastric cancer MGC803 xenograft tumors in nude mice demonstrated that compared with negative control group, mPEG-SC_{20K}-HM-3 36.7 mg/kg played a significant inhibition role in growth of human gastric cancer MGC803. *P<0.05 (there is significant difference compared with negative group).

### EXAMPLE 11

### The Experiment of Tumor Growth Inhibition Induced by mPEG-SC₂₀ₖ-HM-3 on Human Pancreatic Cancer SW-1990 Xenograft Tumors in Nude Mice

Picked the tumor tissue of vigorous growth and was cut into pieces of about 1.5 mm³. Under sterile conditions, the tumor tissue was inoculated subcutaneously on the right of nude mice. Tumor diameter was measured with a Vernier caliper. The animals which had tumors with a size of 100-200 mm³ were randomly divided into groups. The effect of polypeptide on anti-tumor was dynamically observed through the method of measuring tumor diameter. The times of measuring tumor diameter were once 2 days, and meantime, weighed mice. The mice in experimental group were infused polypeptide through tail vein, once 3 days. The mice in the placebo group were injected saline with the same volume. Tumor volume formula:

TV=0.52×a×b²

a, b respectively represent the length and width. According to the result of measurement, calculate the relative tumor volume. Evaluation of antitumor activity is relative tumor proliferation rate T/C (%). The formula is as follows: T/C (%)⁼T_{RTV}/C_{RTV}×100%
T_{RTV}: Treatment group RTV; C_{RTV}: negative control group RTV

TABLE 6
The effect of tumor growth inhibition induced by mPEG-SC₂₀ₖ-HM-3
on human pancreatic cancer SW-1990 xenograft tumors in nude mice

| | | | Primary | | Final | | Tumor | Rate of |
|---|---|---|---|---|---|---|---|---|
| | Dose | Frequency of | weight | Primary | weight | Final | weight | inhibiting |
| Groups | (mg/kg) | administration | (g) | numbers | (g) | numbers | (g) | tumors (%) |
| Negative | — | Once 1 day | 22.30 | 12 | 22.18 | 12 | 1.385 | — |
| control | | | | | | | | |
| 5-Fu | 10 | Once 1 day | 23.17 | 8 | 22.43 | 8 | 0.323 | 76.68% |
| Endostar | 2.5 | Once 1 day | 24.26 | 8 | 23.73 | 8 | 0.932 | 32.71% |
| HM-3 | 1.5 | Once 3 days | 23.40 | 8 | 23.10 | 8 | 0.582 | 57.94% |
| mPEG-SC₂₀ₖ⁻HM-3 | 36.7 | Once 3 days | 24.63 | 8 | 23.32 | 8 | 0.446 | 67.76%* |
| hight | | | | | | | | |
| mPEG-SC₂₀ₖ⁻HM-3 | 18.75 | Once 3 days | 23.33 | 8 | 23.17 | 8 | 0.491 | 64.55% |
| intermediate | | | | | | | | |
| mPEG-SC₂₀ₖ⁻HM-3 | 9.38 | Once 3 days | 23.34 | 8 | 22.67 | 8 | 0.687 | 50.40% |
| low | | | | | | | | |

Results: shown in table 4 and FIG. 5. 5-Fu group: 10 mg/kg, the rate of inhibiting tumor of nude mice with human pancreatic cancer SW-1990 is 76.68%; Endostar group: 2.5 mg/kg, the rate of inhibiting tumor of nude mice with human pancreatic cancer SW-1990 is 32.71%; HM-3 group: 1.5 mg/kg, the rate of inhibiting tumor of nude mice with human pancreatic cancer SW-1990 is 57.94%; The rates of inhibiting tumor of nude mice with human pancreatic cancer SW-1990 in groups of high, intermediate and low dose of polypeptide are 67.76%, 64.55%, 50.40%, respectively.

The results of experiment of tumor growth inhibition induced by mPEG-SC₂₀ₖ-HM-3 on human pancreatic cancer SW-1990 xenograft tumors in nude mice demonstrated that compared with negative control group, mPEG-SC_{20K}-HM-3 36.7 mg/kg played a significant inhibition role in growth of human pancreatic cancer SW-1990. *P<0.05 (there is significant difference compared with negative group).

### EXAMPLE 12

### The Experiment of Tumor Growth Inhibition Induced by mPEG-SC₂₀ₖ-HM-3 on Human Lung Cancer H460 Xenograft Tumors in Nude Mice

Picked the tumor tissue of vigorous growth and was cut into pieces of about 1.5 mm³. Under sterile conditions, the tumor tissue was inoculated subcutaneously on the right of nude mice. Tumor diameter was measured with a vernier caliper. The animals which had tumors with a size of 100-200 mm³ were randomly divided into groups. The effect of polypeptide on anti-tumor was dynamically observed through the method of measuring tumor diameter. The times of measuring tumor diameter were once 2 days, and meantime, weighed mice. The mice in experimental group were infused polypeptide through tail vein, once 3 days. The mice in the placebo group were injected saline with the same volume. Tumor volume formula:

TV=0.52×a×b²

a, b respectively represent the length and width. According to the result of measurement, calculate the relative tumor volume. Evaluation of antitumor activity is relative tumor proliferation rate T/C (%). The formula is as follows: T/C (%)⁻T_{RTV}/C_{RTV}×100%
T_{RTV}: Treatment group RTV; C_{RTV}: negative control group RTV

TABLE 7
The effect of tumor growth inhibition induced by mPEG-SC₂₀ₖ-HM-
3 on human lung cancer H460 xenograft tumors in nude mice

| | | | Primary | | Final | | Tumor | Rate of |
|---|---|---|---|---|---|---|---|---|
| | Dose | Frequency of | weight | Primary | weight | Final | weight | inhibiting |
| Groups | (mg/kg) | administration | (g) | numbers | (g) | numbers | (g) | tumors (%) |
| Negative control | — | Once 1 day | 21.36 | 12 | 21.21 | 12 | 0.794 | — |
| Taxol | 10 | Once 2 days | 22.34 | 8 | 19.58 | 8 | 0.248 | 68.77% |
| Endostar | 2.5 | Once 1 day | 21.08 | 8 | 20.86 | 8 | 0.546 | 31.20% |
| HM-3 | 1.5 | Once 3 days | 21.33 | 8 | 21.11 | 8 | 0.274 | 65.42% |
| mPEG-SC₂₀ₖ-HM-3high | 36.7 | Once 3 days | 21.47 | 8 | 20.79 | 8 | 0.266 | 66.45%* |
| mPEG-SC₂₀ₖ-HM-3intermediate | 18.75 | Once 3 days | 22.38 | 8 | 22.16 | 8 | 0.354 | 55.37% |
| mPEG-SC₂₀ₖ-HM-3low | 9.38 | Once 3 days | 21.28 | 8 | 21.06 | 8 | 0.363 | 54.28% |

Results: shown in table 7 and FIG. 8. Taxol group: 10 mg/kg, the rate of inhibiting tumor of nude mice with human lung cancer H460 is 68.77%; Endostar group: 2.5 mg/kg, the rate of inhibiting tumor of nude mice with human lung cancer H460 is 31.20%; HM-3 group: 1.5 mg/kg, the rate of inhibiting tumor of nude mice with human lung cancer H460 is 65.42%; The rates of inhibiting tumor of nude mice with human lung cancer H460 in groups of high, intermediate and low dose of polypeptide are 66.45%, 55.37%, 54.28%, respectively.

The results of experiment of tumor growth inhibition induced by mPEG-SC₂₀ₖ-HM-3 on human lung cancer H460 xenograft tumors in nude mice demonstrated that compared with negative control group, mPEG-SC_{20K}-HM-3 36.7 mg/kg played a significant inhibition role in growth of human lung cancer H460. *P<0.05(there is significant difference compared with negative group).

### EXAMPLE 13

### The Experiment of Tumor Growth Inhibition Induced by mPEG-SC₂₀ₖ-HM-3 on Human Breast Cancer MDA-MB-231 Xenograft Tumors in Nude Mice

Picked the tumor tissue of vigorous growth and was cut into pieces of about 1.5 mm³. Under sterile conditions, the tumor tissue was inoculated subcutaneously on the right of nude mice. Tumor diameter was measured with a vernier caliper. The animals which had tumors with a size of 100-200 mm³ were randomly divided into groups. The effect of polypeptide on anti-tumor was dynamically observed through the method of measuring tumor diameter. The times of measuring tumor diameter were once 2 days, and meantime, weighed mice. The mice in experimental group were infused polypeptide through tail vein, once 3 days. The mice in the placebo group were injected saline with the same volume. Tumor volume formula: TV=0.52×a×b²

a, b respectively represent the length and width. According to the result of measurement, calculate the relative tumor volume. Evaluation of antitumor activity is relative tumor proliferation rate T/C (%). The formula is as follows: T/C (%)⁻T_{RTV}/C_{RTV}×100%
T_{RTV}: Treatment group RTV; C_{RTV}: negative control group RTV

TABLE 8
The effect of tumor growth inhibition induced by mPEG-SC₂₀ₖ-HM-3
on human breast cancer MDA-MB-231 xenograft tumors in nude mice

| | | | Primary | | Final | | Tumor | Rate of |
|---|---|---|---|---|---|---|---|---|
| | Dose | Frequency of | weight | Primary | weight | Final | weight | inhibiting |
| Groups | (mg/kg) | administration | (g) | numbers | (g) | numbers | (g) | tumors (%) |
| Negative control | — | Once 1 day | 22.32 | 10 | 22.69 | 10 | 1.243 | — |
| Taxol | 10 | Once 2 days | 23.90 | 8 | 23.32 | 8 | 0.335 | 73.05% |
| Endostar | 2.5 | Once 1 day | 24.65 | 8 | 23.03 | 8 | 0.801 | 35.57% |
| HM-3 | 1.5 | Once 3 days | 23.43 | 8 | 23.54 | 8 | 0.533 | 57.14% |
| mPEG-SC₂₀ₖ-HM-3high | 36.7 | Once 3 days | 22.38 | 8 | 22.73 | 8 | 0.459 | 63.05%* |
| mPEG-SC₂₀ₖ-HM-3intermediate | 18.75 | Once 3 days | 23.66 | 8 | 22.34 | 8 | 0.508 | 59.11% |
| mPEG-SC₂₀ₖ-HM-3low | 9.38 | Once 3 days | 23.73 | 8 | 23.49 | 8 | 0.609 | 51.01% |

Results: shown in table 8 and FIG. 9. Taxol group: 10 mg/kg, the rate of inhibiting tumor of nude mice with human breast cancer MDA-MB-231 is 73.05%; Endostar group: 2.5 mg/kg, the rate of inhibiting tumor of nude mice with human breast cancer MDA-MB-231 is 35.57%; HM-3 group: 1.5 mg/kg, the rate of inhibiting tumor of nude mice with human breast cancer MDA-MB-231 is 57.14%; The rates of inhibiting tumor of nude mice with human breast cancer MDA-MB-231 in groups of high, intermediate and low dose of polypeptide are 63.05%, 59.11%, 51.01%, respectively.

The results of experiment of tumor growth inhibition induced by mPEG-SC₂₀ₖ-HM-3 on human breast cancer MDA-MB-231 xenograft tumors in nude mice demonstrated that compared with negative control group, mPEG-SC_{20K}-HM-3 36.7 mg/kg played a significant inhibition role in growth of human breast cancer MDA-MB-231. *P<0.05(there is significant difference compared with negative group).

### EXAMPLE 14

### The Experiment of Tumor Growth Inhibition Induced by mPEG-SC_{20K}-HM-3 on Human Gallbladder Cancer GBC-SD Xenograft Tumors in Nude Mice

Human gallbladder cancer GBC-SD cell lines in logarithmic growth phase were picked and made into 5×10⁷/ml cell suspension of under sterile conditions, and then 0.1 ml suspension was inoculated subcutaneously at the right armpit of nude mice. Tumor diameter was measured with a vernier caliper. The animals which had tumors with a size of 100-200 mm³ were randomly divided into groups. The effect of polypeptide on anti-tumor was dynamically observed through the method of measuring tumor diameter. The times of measuring tumor diameter were once 2 days, and meantime, weighed mice. The mice in experimental group were infused polypeptide through tail vein. The mice in the placebo group were injected saline with the same volume, once 1 day; Taxol group: 10 mg/kg, once 1 week; Endostar group: 2.5 mg/kg, once 1 day; The groups of high, intermediate and low polypeptide: 6, 3, 1.5 mg/kg, respectively, once 1 day. After administration, all mice were sacrificed. Stripped the tumors and weighed.

TABLE 9
The effect of tumor growth inhibition induced by mPEG-SC_{20K}-HM-3
on human gallbladder cancer GBC-SD xenograft tumors in nude mice

| | | | Primary | | Final | | Tumor | Rate of |
|---|---|---|---|---|---|---|---|---|
| | Dose | Frequency of | weight | Primary | weight | Final | weight | inhibiting |
| Groups | (mg/kg) | administration | (g) | numbers | (g) | numbers | (g) | tumors (%) |
| Negative control | — | Once 1 day | 21.21 | 12 | 20.32 | 12 | 1.061 | — |
| Taxol | 10 | Once 2 days | 20.52 | 8 | 20.43 | 8 | 0.232 | 78.13% |
| Endostar | 2.5 | Once 1 day | 20.68 | 8 | 20.45 | 8 | 0.728 | 31.39% |
| HM-3 | 1.5 | Once 3 days | 21.12 | 8 | 20.92 | 8 | 0.450 | 57.59% |
| mPEG-SC₂₀ₖ-HM-3high | 36.7 | Once 3 day | 20.46 | 8 | 20.07 | 8 | 0.409 | 61.45%* |
| mPEG-SC₂₀ₖ-HM-3intermediate | 18.75 | Once 3 day | 21.32 | 8 | 20.56 | 8 | 0.524 | 50.59% |
| mPEG-SC₂₀ₖ-HM-3low | 9.38 | Once 3 day | 21.05 | 8 | 20.58 | 8 | 0.633 | 40.32% |

Results: shown in table 9 and FIG. 10. Taxol group: 10 mg/kg, the rate of inhibiting tumor of nude mice with human gallbladder cancer GBC-SD is78.13%; Endostar group: 2.5 mg/kg, the rate of inhibiting tumor of nude mice with human gallbladder cancer GBC-SD is 31.39%; HM-3 group: 1.5 mg/kg, the rate of inhibiting tumor of nude mice with human gallbladder cancer GBC-SD is 57.59%; The rates of inhibiting tumor of nude mice with human gallbladder cancer GBC-SD in groups of high, intermediate and low dose of polypeptide are 61.45%, 50.59%, 40.32%, respectively.

The results of experiment of tumor growth inhibition induced by mPEG-SC₂₀ₖ-HM-3 on human gallbladder cancer GBC-SD xenograft tumors in nude mice demonstrated that compared with negative control group, mPEG-SC_{20K}-HM-3 36.7 mg/kg played a significant inhibition role in growth of human gallbladder cancer GBC-SD. *P<0.05 (there is significant difference compared with negative group).

### EXAMPLE 15

### The Experiment of Tumor Growth Inhibition Induced by mPEG-SC_{20K}-HM-3 on Human Kidney Cancer A498 Xenograft Tumors in Nude Mice

Human kidney cancer A498 cell lines in logarithmic growth phase were picked and made into 5×10⁷/ml cell suspension of under sterile conditions, and then 0.1 ml suspension was inoculated subcutaneously at the right armpit of nude mice. Tumor diameter was measured with a vernier caliper. The animals which had tumors with a size of 100-200 mm³ were randomly divided into groups. The effect of polypeptide on anti-tumor was dynamically observed through the method of measuring tumor diameter. The times of measuring tumor diameter were once 2 days, and meantime, weighed mice. The mice in experimental group were infused polypeptide through tail vein. The mice in the placebo group were injected saline with the same volume, once 1 day; Taxol group: 10 mg/kg, once 1 week; Endostar group: 2.5 mg/kg, once 1 day; The groups of high, intermediate and low polypeptide: 6, 3, 1.5 mg/kg, respectively, once 1 day. After administration, all mice were sacrificed. Stripped the tumors and weighed.

TABLE 10
The effect of tumor growth inhibition induced by mPEG-SC_{20K}-HM-
3 on human kidney cancer A498 xenograft tumors in nude mice

| | | | Primary | | Final | | Tumor | Rate of |
|---|---|---|---|---|---|---|---|---|
| | Dose | Frequency of | weight | Primary | weight | Final | weight | inhibiting |
| Groups | (mg/kg) | administration | (g) | numbers | (g) | numbers | (g) | tumors (%) |
| Negative control | — | Once 1 day | 21.38 | 10 | 21.11 | 12 | 0.944 | — |
| Taxol | 10 | Once 2 days | 21.23 | 6 | 21.15 | 6 | 0.242 | 74.32% |
| Endostar | 2.5 | Once 1 day | 21.43 | 6 | 21.66 | 6 | 0.656 | 30.51% |
| HM-3 | 1.5 | Once 3 days | 20.47 | 8 | 20.88 | 8 | 0.427 | 54.77% |
| mPEG-SC₂₀ₖ⁻HM-3high | 36.7 | Once 3 days | 20.00 | 6 | 20.23 | 6 | 0.424 | 55.11%* |
| mPEG-SC₂₀ₖ⁻HM-3intermediate | 18.75 | Once 3 days | 21.34 | 6 | 20.48 | 6 | 0.501 | 46.95% |
| mPEG-SC₂₀ₖ⁻HM-3low | 9.38 | Once 3 days | 20.35 | 6 | 20.76 | 6 | 0.576 | 39.00% |

Results: shown in table 10 and FIG. 11. Taxol group: 10 mg/kg, the rate of inhibiting tumor of nude mice with human kidney cancer A498 is 84.32%; Endostar group: 2.5 mg/kg, the rate of inhibiting tumor of nude mice with human kidney cancer A498 is 30.51%; HM-3 group: 1.5 mg/kg, the rate of inhibiting tumor of nude mice with human kidney cancer A498 is 54.77%; The rates of inhibiting tumor of nude mice with human kidney cancer A498 in groups of high, intermediate and low dose of polypeptide are 55.11%, 46.95%, 39.00%, respectively.

The results of experiment of tumor growth inhibition induced by mPEG-SC₂₀ₖ-HM-3 on human kidney cancer A498 xenograft tumors in nude mice demonstrated that compared with negative control group, mPEG-SC_{20K}-HM-3 36.7 mg/kg played a significant inhibition role in growth of human kidney cancer A498. *P<0.05(there is significant difference compared with negative group).

### EXAMPLE 16

### The Experiment of Tumor Growth Inhibition Induced by mPEG-SC₂₀ₖ-HM-3 on Human Colon Cancer HT-29 Xenograft Tumors in Nude Mice

Human colon cancer HT-29 cell lines in logarithmic growth phase were picked, and made into 5×10⁷/ml cell suspension of under sterile conditions, and then 0.1 ml suspension was inoculated subcutaneously at the right armpit of nude mice. Tumor diameter was measured with a vernier caliper. The animals which had tumors with a size of 100-200 mm³ were randomly divided into groups. The effect of polypeptide on anti-tumor was dynamically observed through the method of measuring tumor diameter. The times of measuring tumor diameter were once 2 days, and meantime, weighed mice. The mice in experimental group were infused polypeptide through tail vein. The mice in the placebo group were injected saline with the same volume, once 1 day; Taxol group: 10 mg/kg, once 1 week; Endostar group: 2.5 mg/kg, once 1 day; The groups of high, intermediate and low polypeptide: 6, 3, 1.5 mg/kg, respectively, once 1 day. After administration, all mice were sacrificed. Stripped the tumors and weighed.

TABLE 11
The effect of tumor growth inhibition induced by mPEG-SC₂₀ₖ-HM-
3 on human colon cancer HT-29 xenograft tumors in nude mice

| | | | Primary | | Final | | Tumor | Rate of |
|---|---|---|---|---|---|---|---|---|
| | Dose | Frequency of | weight | Primary | weight | Final | weight | inhibiting |
| Groups | (mg/kg) | administration | (g) | numbers | (g) | numbers | (g) | tumors (%) |
| Negative | — | Once 1 day | 23.94 | 12 | 23.67 | 12 | 1.120 | — |
| control | | | | | | | | |
| Taxol | 10 | Once 2 days | 23.43 | 8 | 22.84 | 6 | 0.346 | 69.11% |
| Endostar | 2.5 | Once 1 day | 23.23 | 8 | 22.81 | 8 | 0.745 | 33.48% |
| HM-3 | 1.5 | Once 3 days | 24.11 | 12 | 23.15 | 12 | 0.531 | 52.59% |
| mPEG-SC₂₀ₖ-HM-3 | 36.7 | Once 3 days | 23.45 | 8 | 22.68 | 8 | 0.493 | 55.98%* |
| high | | | | | | | | |
| mPEG-SC₂₀ₖ-HM-3 | 18.75 | Once 3 days | 24.61 | 8 | 25.36 | 8 | 0.611 | 45.45% |
| intermediate | | | | | | | | |
| mPEG-SC₂₀ₖ-HM-3 | 9.38 | Once 3 days | 24.29 | 8 | 23.17 | 8 | 0.705 | 37.05% |
| low | | | | | | | | |

Results: shown in table 11 and FIG. 12. Taxol group: 10 mg/kg, the rate of inhibiting tumor of nude mice with human colon cancer HT-29 is 69.11%; Endostar group: 2.5 mg/kg, the rate of inhibiting tumor of nude mice with human colon cancer HT-29 is 33.48%; HM-3 group: 1.5 mg/kg, the rate of inhibiting tumor of nude mice with human colon cancer HT-29 is 52.59%; The rates of inhibiting tumor of nude mice with human colon cancer HT-29 in groups of high, intermediate and low dose of polypeptide are 55.98%, 45.45%, 37.05%, respectively.

The results of experiment of tumor growth inhibition induced by mPEG-SC₂₀ₖ-HM-3 on human colon cancer HT-29 xenograft tumors in nude mice demonstrated that compared with negative control group, mPEG-SC_{20K}-HM-3 36.7 mg/kg played a significant inhibition role in growth of human colon cancer HT-29. *P<0.05(there is significant difference compared with negative group).

### EXAMPLE 17

### The Experiment of Tumor Growth Inhibition Induced by mPEG-SC₂₀ₖ-HM-3 on Human Ovarian Cancer SK-OV-3 Xenograft Tumors in Nude Mice

Human ovarian cancer SK-OV-3 cell lines in logarithmic growth phase were picked, and made into 5×10⁷/ml cell suspension of under sterile conditions, and then 0.1 ml suspension was inoculated subcutaneously at the right armpit of nude mice. Tumor diameter was measured with a vernier caliper. The animals which had tumors with a size of 100-200 mm³ were randomly divided into groups. The effect of polypeptide on anti-tumor was dynamically observed through the method of measuring tumor diameter. The times of measuring tumor diameter were once 2 days, and meantime, weighed mice. The mice in experimental group were infused polypeptide through tail vein. The mice in the placebo group were injected saline with the same volume, once 1 day; Cisplatin group: 10 mg/kg, once 1 week; Endostar group: 2.5 mg/kg, once 1 day; The groups of high, intermediate and low polypeptide: 6, 3, 1.5 mg/kg, respectively, once 1 day. After administration, all mice were sacrificed. Stripped the tumors and weighed.

TABLE 12
The effect of tumor growth inhibition induced by mPEG-SC₂₀ₖ-HM-
3 on human ovarian cancer SK-OV-3 xenograft tumors in nude mice

| | | | Primary | | Final | | Tumor | Rate of |
|---|---|---|---|---|---|---|---|---|
| | Dose | Frequency of | weight | Primary | weight | Final | weight | inhibiting |
| Groups | (mg/kg) | administration | (g) | numbers | (g) | numbers | (g) | tumors (%) |
| Negative | — | Once 1 day | 23.51 | 12 | 22.03 | 12 | 1.265 | — |
| control | | | | | | | | |
| Cisplatin | 10 | Twice 1 week | 22.43 | 8 | 22.19 | 6 | 0.302 | 76.13% |
| Endostar | 2.5 | Once 1 day | 22.94 | 8 | 22.74 | 8 | 0.860 | 31.98% |
| HM-3 | 1.5 | Once 3 days | 22.90 | 8 | 22.85 | 8 | 0.639 | 49.49% |
| mPEG-SC₂₀ₖ-HM-3 | 36.7 | Once 3 days | 23.53 | 8 | 22.35 | 8 | 0.627 | 50.40%* |
| high | | | | | | | | |
| mPEG-SC₂₀ₖ-HM-3 | 18.75 | Once 3 days | 23.66 | 8 | 22.29 | 8 | 0.700 | 44.62% |
| intermediate | | | | | | | | |
| mPEG-SC₂₀ₖ-HM-3 | 9.38 | Once 3 days | 22.89 | 8 | 22.74 | 8 | 0.730 | 42.33% |
| low | | | | | | | | |

Results: shown in table 12 and FIG. 13. Cisplatin group: 10 mg/kg, the rate of inhibiting tumor of nude mice with human ovarian cancer SK-OV-3 is 76.13%; Endostar group: 2.5 mg/kg, the rate of inhibiting tumor of nude mice with human ovarian cancer SK-OV-3 is 31.98%; HM-3 group: 1.5 mg/kg, the rate of inhibiting tumor of nude mice with human ovarian cancer SK-OV-3 is 49.49%; The rates of inhibiting tumor of nude mice with human ovarian cancer SK-OV-3 in groups of high, intermediate and low dose of polypeptide are 50.40%, 44.62%, 42.33%, respectively.

Therefore, the results of experiment of tumor growth inhibition induced by mPEG-SC₂₀ₖ-HM-3 on human ovarian cancer SK-OV-3 xenograft tumors in nude mice demonstrated that compared with negative control group, mPEG-SC_{20K}-HM-3 36.7 mg/kg played a significant inhibition role in growth of human ovarian cancer SK-OV-3. *P<0.05(there is significant difference compared with negative group).

### EXAMPLE 18

### The Experiment of Tumor Growth Inhibition Induced by mPEG-SC_{20K}-HM-3 on Human Endometrial Cancer HHUA Xenograft Tumors in Nude Mice

Human endometrial cancer HHUA cell lines in logarithmic growth phase were picked, and made into 5×10⁷/ml cell suspension of under sterile conditions, and then 0.1 ml suspension was inoculated subcutaneously at the right armpit of nude mice. Tumor diameter was measured with a vernier caliper. The animals which had tumors with a size of 100-200 mm³ were randomly divided into groups. The effect of polypeptide on anti-tumor was dynamically observed through the method of measuring tumor diameter. The times of measuring tumor diameter were once 2 days, and meantime, weighed mice. The mice in experimental group were infused polypeptide through tail vein. The mice in the placebo group were injected saline with the same volume, once 1 day; Taxol group: 10 mg/kg, once 1 week; Endostar group: 2.5 mg/kg, once 1 day; The groups of high, intermediate and low polypeptide: 6, 3, 1.5 mg/kg, respectively, once 1 day. After administration, all mice were sacrificed. Stripped the tumors and weighed.

TABLE 13
The effect of tumor growth inhibition induced by mPEG-SC_{20K}-HM-
3 on human endometrial cancer HHUA xenograft tumors in nude mice

| | | | Primary | | Final | | Tumor | Rate of |
|---|---|---|---|---|---|---|---|---|
| | Dose | Frequency of | weight | Primary | weight | Final | weight | inhibiting |
| Groups | (mg/kg) | administration | (g) | numbers | (g) | numbers | (g) | tumors (%) |
| Negative | — | Once 1 day | 24.51 | 12 | 23.03 | 12 | 1.208 | — |
| control | | | | | | | | |
| Taxol | 10 | Twicw 1 week | 23.43 | 8 | 22.19 | 6 | 0.295 | 75.55% |
| Endostar | 2.5 | Once 1 day | 22.98 | 8 | 22.74 | 8 | 0.785 | 34.98% |
| HM-3 | 1.5 | Once 3 days | 22.94 | 8 | 22.85 | 8 | 0.550 | 54.49% |
| mPEG-SC₂₀ₖ-HM-3 | 36.7 | Once 3 days | 23.98 | 8 | 22.39 | 8 | 0.453 | 62.47%* |
| high | | | | | | | | |
| mPEG-SC₂₀ₖ-HM-3 | 18.75 | Once 3 days | 23.76 | 8 | 22.37 | 8 | 0.560 | 53.65% |
| itermediate | | | | | | | | |
| mPEG-SC₂₀ₖ-HM-3 | 9.38 | Once 3 days | 22.89 | 8 | 22.66 | 8 | 0.587 | 51.38% |
| low | | | | | | | | |

Results: shown in table 13 and FIG. 14. Taxol group: 10 mg/kg, the rate of inhibiting tumor of nude mice with human endometrial cancer HHUA is 75.55%; Endostar group: 2.5 mg/kg, the rate of inhibiting tumor of nude mice with human endometrial cancer HHUA is 34.98%; HM-3 group: 1.5 mg/kg, the rate of inhibiting tumor of nude mice with human endometrial cancer HHUA is 54.49%; The rates of inhibiting tumor of nude mice with human endometrial cancer HHUA in groups of high, intermediate and low dose of polypeptide are 62.47%, 53.65%, 51.38%, respectively.

Therefore, the results of experiment of tumor growth inhibition induced by mPEG-SC₂₀ₖ-HM-3 on human endometrial cancer HHUA xenograft tumors in nude mice demonstrated that compared with negative control group, mPEG-SC_{20K}-HM-3 36.7 mg/kg played a significant inhibition role in growth of human endometrial cancer HHUA. *P<0.05(there is significant difference compared with negative group).

### EXAMPLE 19

### The Experiment of Tumor Growth Inhibition Induced by mPEG-SC_{20K}-HM-3 on Human Cervical Cancer HeLa Xenograft Tumors in Nude Mice

Human cervical cancer HeLa cell lines in logarithmic growth phase were picked, and made into 5×10⁷/ml cell suspension of under sterile conditions, and then 0.1 ml suspension was inoculated subcutaneously at the right armpit of nude mice. Tumor diameter was measured with a vernier caliper. The animals which had tumors with a size of 100-200 mm³ were randomly divided into groups. The effect of polypeptide on anti-tumor was dynamically observed through the method of measuring tumor diameter. The times of measuring tumor diameter were once 2 days, and meantime, weighed mice. The mice in experimental group were infused polypeptide through tail vein. The mice in the placebo group were injected saline with the same volume, once 1 day; Taxol group: 10 mg/kg, once 1 week; Endostar group: 2.5 mg/kg, once 1 day; The groups of high, intermediate and low polypeptide: 6, 3, 1.5 mg/kg, respectively, once 1 day. After administration, all mice were sacrificed. Stripped the tumors and weighed.

TABLE 14
The effect of tumor growth inhibition induced by mPEG-SC_{20K}-HM-
3 on human cervical cancer HeLa xenograft tumors in nude mice

| | | | Primary | | Final | | Tumor | Rate of |
|---|---|---|---|---|---|---|---|---|
| | Dose | Frequency of | weight | Primary | weight | Final | weight | inhibiting |
| Groups | (mg/kg) | administration | (g) | numbers | (g) | numbers | (g) | tumors (%) |
| Negative | — | Once 1 day | 23.11 | 8 | 23.35 | 8 | 1.236 | — |
| control | | | | | | | | |
| Taxol | 10 | Once 2 days | 24.36 | 6 | 23.47 | 6 | 0.428 | 65.37% |
| Endostar | 2.5 | Once 1 day | 23.54 | 6 | 23.28 | 6 | 0.796 | 35.57% |
| HM-3 | 1.5 | Once 3 days | 24.11 | 6 | 23.86 | 6 | 0.528 | 57.25% |
| mPEG-SC₂₀ₖ-HM-3 | 36.7 | Once 3 days | 23.76 | 6 | 23.05 | 6 | 0.222 | 82.07%* |
| high | | | | | | | | |
| mPEG-SC₂₀ₖ-HM-3 | 18.75 | Once 3 days | 24.80 | 6 | 24.43 | 6 | 0.320 | 74.11% |
| itermediate | | | | | | | | |
| mPEG-SC₂₀ₖ-HM-3 | 9.38 | Once 3 days | 24.10 | 6 | 23.85 | 6 | 0.453 | 63.32% |
| low | | | | | | | | |

Results: shown in table 14 and FIGS. 15,16. Taxol group: 10 mg/kg, the rate of inhibiting tumor of nude mice with human cervical cancer HeLa is 65.37%; Endostar group: 2.5 mg/kg, the rate of inhibiting tumor of nude mice with human cervical cancer HeLa is 35.57%; HM-3 group: 1.5 mg/kg, the rate of inhibiting tumor of nude mice with human cervical cancer HeLa is 57.25%; The rates of inhibiting tumor of nude mice with human cervical cancer HeLa in groups of high, intermediate and low dose of polypeptide are 82.07%, 74.11%, 63.32%, respectively.

Therefore, the results of experiment of tumor growth inhibition induced by mPEG-SC₂₀ₖ-HM-3 on human cervical cancer HeLa xenograft tumors in nude mice demonstrated that compared with negative control group, mPEG-SC_{20K}-HM-3 36.7 mg/kg played a significant inhibition role in growth of human cervical cancer HeLa. *P<0.05(there is significant difference compared with negative group)

### EXAMPLE 20

### The Experiment of Tumor Growth Inhibition Induced by mPEG-SC_{20K}-HM-3 on Human Prostate Cancer DU-145 Xenograft Tumors in Nude Mice

Human prostate cancer DU-145 cell lines in logarithmic growth phase were picked, and made into 5×10⁷/ml cell suspension of under sterile conditions, and then 0.1 ml suspension was inoculated subcutaneously at the right armpit of nude mice. Tumor diameter was measured with a vernier caliper. The animals which had tumors with a size of 100-200 mm³ were randomly divided into groups. The effect of polypeptide on anti-tumor was dynamically observed through the method of measuring tumor diameter. The times of measuring tumor diameter were once 2 days, and meantime, weighed mice. The mice in experimental group were infused polypeptide through tail vein. The mice in the placebo group were injected saline with the same volume, once 1 day; Cisplatin group: 10 mg/kg, once 1 week; Endostar group: 2.5 mg/kg, once 1 day; The groups of high, intermediate and low polypeptide: 6, 3, 1.5 mg/kg, respectively, once 1 day. After administration, all mice were sacrificed. Stripped the tumors and weighed.

TABLE 15
The effect of tumor growth inhibition induced by mPEG-SC_{20K}-HM-
3 on human prostate cancer DU-145 xenograft tumors in nude mice

| | | | Primary | | Final | | Tumor | Rate of |
|---|---|---|---|---|---|---|---|---|
| | Dose | Frequency of | weight | Primary | weight | Final | weight | inhibiting |
| Groups | (mg/kg) | administration | (g) | numbers | (g) | numbers | (g) | tumors (%) |
| Negative | — | Once 1 day | 24.12 | 12 | 23.65 | 12 | 1.854 | — |
| control | | | | | | | | |
| Taxol | 10 | Twice a week | 24.57 | 8 | 23.81 | 6 | 0.545 | 70.60% |
| Endostar | 2.5 | Once 1 day | 23.08 | 8 | 23.25 | 8 | 1.274 | 31.25% |
| HM-3 | 1.5 | Once 3 days | 24.77 | 8 | 23.64 | 8 | 0.809 | 56.36% |
| mPEG-SC₂₀ₖ-HM-3 | 36.7 | Once 3 days | 23.26 | 8 | 23.08 | 8 | 0.417 | 77.46%* |
| high | | | | | | | | |
| mPEG-SC₂₀ₖ-HM-3 | 18.75 | Once 3 days | 24.38 | 8 | 23.61 | 8 | 0.603 | 67.48% |
| itermediate | | | | | | | | |
| mPEG-SC₂₀ₖ-HM-3 | 9.38 | Once 3 days | 24.41 | 8 | 23.37 | 8 | 0.781 | 57.87% |
| low | | | | | | | | |

Results: shown in table 15 and FIG. 17. Cisplatin group: 10 mg/kg, the rate of inhibiting tumor of nude mice with human prostate cancer DU-145 is 70.60%; Endostar group: 2.5 mg/kg, the rate of inhibiting tumor of nude mice with human prostate cancer DU-145 is 31.25%; HM-3 group: 1.5 mg/kg, the rate of inhibiting tumor of nude mice with human prostate cancer DU-145 is 56.36%; The rates of inhibiting tumor of nude mice with human prostate cancer DU-145 in groups of high, intermediate and low dose of polypeptide are 77.46%, 67.48%, 57.87%, respectively.

Therefore, the results of experiment of tumor growth inhibition induced by mPEG-SC₂₀ₖ-HM-3 on human prostate cancer DU-145 xenograft tumors in nude mice demonstrated that compared with negative control group, mPEG-SC_{20K}-HM-3 36.7 mg/kg played a significant inhibition role in growth of human prostate cancer DU-145. *P<0.05(there is significant difference compared with negative group).

### EXAMPLE 21

### The Experiment of Tumor Growth Inhibition Induced by mPEG-SC_{20K}-HM-3 on Human Bladder Cancer HT1376 Xenograft Tumors in Nude Mice

Human bladder cancer HT1376 cell lines in logarithmic growth phase were picked, and made into 5×10⁷/ml cell suspension of under sterile conditions, and then 0.1 ml suspension was inoculated subcutaneously at the right armpit of nude mice. Tumor diameter was measured with a vernier caliper. The animals which had tumors with a size of 100-200 mm³ were randomly divided into groups. The effect of polypeptide on anti-tumor was dynamically observed through the method of measuring tumor diameter. The times of measuring tumor diameter were once 2 days, and meantime, weighed mice. The mice in experimental group were infused polypeptide through tail vein. The mice in the placebo group were injected saline with the same volume, once 1 day; Taxol group: 10 mg/kg, once 1 week; Endostar group: 2.5 mg/kg, once 1 day; The groups of high, intermediate and low polypeptide: 6, 3, 1.5 mg/kg, respectively, once 1 day. After administration, all mice were sacrificed. Stripped the tumors and weighed.

TABLE 16
The effect of tumor growth inhibition induced by mPEG-SC_{20K}-HM-
3 on human bladder cancer HT1376 xenograft tumors in nude mice

| | | | Primary | | Final | | Tumor | Rate of |
|---|---|---|---|---|---|---|---|---|
| | Dose | Frequency of | weight | Primary | weight | Final | weight | inhibiting |
| Groups | (mg/kg) | administration | (g) | numbers | (g) | numbers | (g) | tumors (%) |
| Negative | — | Once 1 day | 21.71 | 10 | 21.32 | 10 | 1.018 | — |
| control | | | | | | | | |
| Taxol | 10 | Once 2 days | 21.39 | 6 | 21.65 | 5 | 0.327 | 67.88% |
| Endostar | 2.5 | Once 1 day | 21.52 | 6 | 21.36 | 6 | 0.698 | 31.43% |
| HM-3 | 1.5 | Once 3 days | 22.84 | 6 | 22.30 | 6 | 0.504 | 50.49% |
| mPEG-SC₂₀ₖ-HM-3 | 36.7 | Once 3 days | 22.27 | 6 | 21.59 | 6 | 0.372 | 63.42%* |
| high | | | | | | | | |
| mPEG-SC₂₀ₖ-HM-3 | 18.75 | Once 3 days | 21.58 | 6 | 21.49 | 6 | 0.466 | 54.24% |
| itermediate | | | | | | | | |
| mPEG-SC₂₀ₖ- HM-3 | 9.38 | Once 3 days | 22.76 | 6 | 22.23 | 6 | 0.546 | 46.39% |
| low | | | | | | | | |

Results: shown in table 16 and FIGS. 18,19. Taxol group: 10 mg/kg, the rate of inhibiting tumor of nude mice with human bladder cancer HT1376 is 67.88%; Endostar group: 2.5 mg/kg, the rate of inhibiting tumor of nude mice with human bladder cancer HT1376 is 31.43%; HM-3 group: 1.5 mg/kg, the rate of inhibiting tumor of nude mice with human bladder cancer HT1376 is 50.49%; The rates of inhibiting tumor of nude mice with human bladder cancer HT1376 in groups of high, intermediate and low dose of polypeptide are 63.42%, 54.24%, 46.39%, respectively.

Therefore, the results of experiment of tumor growth inhibition induced by mPEG-SC₂₀ₖ-HM-3 on human bladder cancer HT1376 xenograft tumors in nude mice demonstrated that compared with negative control group, mPEG-SC_{20K}-HM-3 36.7 mg/kg played a significant inhibition role in growth of human bladder cancer HT1376. *P<0.05(there is significant difference compared with negative group).

### EXAMPLE 22

### The Experiment of Tumor Growth Inhibition Induced by mPEG-SC_{20K}-HM-3 on Human Testicular Cancer 5637 Xenograft Tumors in Nude Mice

Human testicular cancer 5637 cell lines in logarithmic growth phase were picked, and made into 5×10⁷/ml cell suspension of under sterile conditions, and then 0.1 ml suspension was inoculated subcutaneously at the right armpit of nude mice. Tumor diameter was measured with a vernier caliper. The animals which had tumors with a size of 100-200 mm³ were randomly divided into groups. The effect of polypeptide on anti-tumor was dynamically observed through the method of measuring tumor diameter. The times of measuring tumor diameter were once 2 days, and meantime, weighed mice. The mice in experimental group were infused polypeptide through tail vein. The mice in the placebo group were injected saline with the same volume, once 1 day; Cisplatin group: 10 mg/kg, once 1 week; Endostar group: 2.5 mg/kg, once 1 day; The groups of high, intermediate and low polypeptide: 6, 3, 1.5 mg/kg, respectively, once 1 day. After administration, all mice were sacrificed. Stripped the tumors and weighed.

TABLE 17
The effect of tumor growth inhibition induced by mPEG-SC_{20K}-HM-
3 on human testicular cancer 5637 xenograft tumors in nude mice

| | | | Primary | | Final | | Tumor | Rate of |
|---|---|---|---|---|---|---|---|---|
| | Dose | Frequency of | weight | Primary | weight | Final | weight | inhibiting |
| Groups | (mg/kg) | administration | (g) | numbers | (g) | numbers | (g) | tumors (%) |
| Negative | — | Once 1 day | 24.08 | 10 | 23.91 | 10 | 2.015 | — |
| control | | | | | | | | |
| Taxol | 10 | Twice 1 week | 24.73 | 6 | 24.46 | 6 | 0.590 | 70.74% |
| Endostar | 2.5 | Once 1 day | 25.07 | 6 | 24.89 | 6 | 1.410 | 30.02% |
| HM-3 | 1.5 | Once 3 days | 25.66 | 6 | 25.15 | 6 | 0.82 | 59.40% |
| mPEG-SC₂₀ₖ-HM-3 | 36.7 | Once 3 days | 25.45 | 6 | 24.18 | 6 | 0.802 | 60.20%* |
| high | | | | | | | | |
| mPEG-SC₂₀ₖ-HM-3 | 18.75 | Once 3 days | 24.24 | 6 | 24.19 | 6 | 0.981 | 51.32% |
| itermediate | | | | | | | | |
| mPEG-SC₂₀ₖ-HM-3 | 9.38 | Once 3 days | 25.74 | 6 | 25.18 | 6 | 0.988 | 50.99% |
| low | | | | | | | | |

Results: shown in table 17 and FIG. 20. Cisplatin group: 10 mg/kg, the rate of inhibiting tumor of nude mice with human testicular cancer 5637 is70.74%; Endostar group: 2.5 mg/kg, the rate of inhibiting tumor of nude mice with human testicular cancer 5637 is 30.02%; HM-3 group: 1.5 mg/kg, the rate of inhibiting tumor of nude mice with human testicular cancer 5637 is 39.40%; The rates of inhibiting tumor of nude mice with human testicular cancer 5637 in groups of high, intermediate and low dose of polypeptide are 60.20%, 51.32%, 50.99%, respectively.

Therefore, the results of experiment of tumor growth inhibition induced by mPEG-SC₂₀ₖ-HM-3 on human testicular cancer 5637 xenograft tumors in nude mice demonstrated that compared with negative control group, mPEG-SC_{20K}-HM-3 36.7 mg/kg played a significant inhibition role in growth of human testicular cancer 5637. *P<0.05(there is significant difference compared with negative group).

### EXAMPLE 25

### The Experiment of Tumor Growth Inhibition Induced by mPEG-SC_{20K}-HM-3 on Sarcoma HT-1080 Xenograft Tumors in Nude Mice

Sarcoma HT-1080 cell lines in logarithmic growth phase were picked, and made into 5×10⁷/ml cell suspension of under sterile conditions, and then 0.1 ml suspension was inoculated subcutaneously at the right armpit of nude mice. Tumor diameter was measured with a vernier caliper. The animals which had tumors with a size of 100-200 mm³ were randomly divided into groups. The effect of polypeptide on anti-tumor was dynamically observed through the method of measuring tumor diameter. The times of measuring tumor diameter were once 2 days, and meantime, weighed mice. The mice in experimental group were infused polypeptide through tail vein. The mice in the placebo group were injected saline with the same volume, once 1 day; Cyclophosphamide group: 15 mg/kg, once 1 week; Polypeptide group: 3 mg/kg, once 1 day. After administration, all mice were sacrificed. Stripped the tumors and weighed.

TABLE 18
The effect of tumor growth inhibition induced by
mPEG-SC_{20K}-HM-3 on sarcoma HT-1080 xenograft tumors in nude mice

| | | | Primary | | Final | | Tumor | Rate of |
|---|---|---|---|---|---|---|---|---|
| | Dose | Frequency of | weight | Primary | weight | Final | weight | inhibiting |
| Groups | (mg/kg) | administration | (g) | numbers | (g) | numbers | (g) | tumors (%) |
| Negative control | — | Once 1 day | 23.13 | 8 | 22.81 | 8 | 1.938 | — |
| Cyclophosphamide | 15 | Once 2 days | 24.09 | 8 | 23.85 | 8 | 0.501 | 74.15% |
| HM-3 | 1.5 | Once 3 days | 24.20 | 8 | 23.89 | 8 | 0.790 | 59.24% |
| mPEG-SC₂₀ₖ-HM-3 | 36.7 | Once 3 days | 23.57 | 8 | 23.26 | 8 | 0.678 | 65.04%* |

Results: shown in table 18 and FIG. 21. Cyclophosphamide group: 10 mg/kg, the rate of inhibiting tumor of nude mice with sarcoma HT-1080 is74.15%; HM-3 group: 1.5 mg/kg, the rate of inhibiting tumor of nude mice with sarcoma HT-1080 is 59.24%; The rate of inhibiting tumor of nude mice with human sarcoma HT-1080 in polypeptide group is 65.04%.

Therefore, the results of experiment of tumor growth inhibition induced by mPEG-SC₂₀ₖ-HM-3 on sarcoma HT-1080 xenograft tumors in nude mice demonstrated that compared with negative control group, mPEG-SC_{20K}-HM-3 36.7 mg/kg played a significant inhibition role in growth of sarcoma HT-1080. *P<0.05 (there is significant difference compared with negative group).

1. A composition comprising:
an integrin inhibitor of SEQ ID NO 4, wherein the methoxypolyethylene glycol succinimidyl carbonate (mPEG-SC) of SEQ ID NO 4 has a molecular weight between 500-20000.

2. The composition of claim 1, wherein the molecular weight of mPEG-SC is 20000.

3. A method for treating a solid tumor comprising the steps of:
administering an integrin inhibitor of SEQ ID NO 4.

4. The method of claim 3, wherein the methoxypolyethylene glycol succinimidyl carbonate (mPEG-SC) of the SEQ ID NO 4 has a molecular weight between 500 and 20000.

5. The method of claim 3, wherein the solid tumor is present on the stomach, skin, head, neck, thyroid, pancreas, lung, esophagus, breast, kidney, gall bladder, colon, rectum, ovary, uterus, cervix, prostate, bladder, testicles.

6. The method of claim 5, wherein the solid tumor is testicular primary cancer, testicular secondary cancer, or sarcoma.


SEQUENCE LISTING (4 sequences)

SEQ ID NO: 1 - protein, 18 aa, Artificial Sequence
  integrin antagonist polypeptide
IVRRADRAAVPGGGGRGD

SEQ ID NO: 2 - protein, 7 aa, Homo sapiens
GGGGRGD

SEQ ID NO: 3 - protein, 11 aa, Artificial Sequence
  angiogenesis inhibition sequence
IVRRADRAAVP

SEQ ID NO: 4 - protein, 18 aa, Artificial Sequence
  polyethylene glycol modified integrin inhibitor
IVRRADRAAVPGGGGRGD